(19) United States
(12) Patent Application Publication
(10) Pub. No.: US 2026/0053467 A1
(43) Pub. Date: Feb. 26, 2026

(54) Automating Ultrasound Image Capture Platform Using Robotics and Artificial Intelligence for eFAST Triage Procedures

(71) Applicant: The Government of The United States, as represented by The Director of the Defense Health Agency, Fort Detrick, MD (US)

(72) Inventors: Eric J. Snider, San Antonio, TX (US); Sofia I. Hernández Torres, San Antonio, TX (US); Krysta-Lynn H. Amezcua, San Antonio, TX (US); James P. Collier, III, San Antonio, TX (US)

(73) Assignee: The Government of The United States, as represented by The Director of the Defense Health Agency, Fort Detrick, MD (US)

(21) Appl. No.: 19/307,648

(22) Filed: Aug. 22, 2025

Related U.S. Application Data
(60) Provisional application No. 63/686,839, filed on Aug. 25, 2024.
(60) Provisional application No. 63/686,836, filed on Aug. 25, 2024.

(51) Int. Cl. A61B 8/00; A61B 8/08

(52) U.S. Cl. CPC A61B 8/4218; A61B 8/085; A61B 8/0875; A61B 8/4281; A61B 8/463; A61B 8/467; A61B 8/54

(57) ABSTRACT

A method and system for obtaining one or more ultrasound images for use in an eFAST examination. A robotic arm is moved along a patient's body while sending images to a controller; the images are analyzed for anatomical landmarks to identify measurement areas; instructions are sent from the controller to the robotic arm to direct the robotic arm to the measurement areas; and an ultrasound transducer is pressed against the patient's skin to capture an ultrasound image upon reaching the measurement area.

### PRIORITY CLAIM

This application claims the benefit of provisional application Ser. No. 63/686,839 filed Aug. 25, 2024 and titled “Automated Ultrasound Image Capture Platform Using Robotics and Artificial Intelligence for eFAST Triage Procedures” and provisional application Ser. No. 63/686,836 filed Aug. 25, 2024 and titled “Automating Ultrasound eFAST Triage Using Artificial Intelligent Models” the entire contents of which are hereby incorporated by reference.

### STATEMENT OF GOVERNMENT INTEREST

The invention described herein may be manufactured, used and licensed by or for the United States Government.

### BACKGROUND

For emergency medicine, triage is a fundamental step for identifying injury severity and prioritize use of resources when treating casualties. This is especially critical when resources are constrained, such as in remote medical situations faced in rural areas or in military medicine. This need only increases in mass casualty situations where triaging tools are needed to characterize injuries to quantify the level of care required for each patient to alleviate the stress on resources in emergency, pre-hospital settings. While physical examinations and basic vital signs are frequently used for high level casualty triage, medical imaging can provide a higher granularity for triage which will be essential on the future battlefield and other remote medical environments. A wide range of clinical imaging options are available in civilian settings for routine emergency medicine; however, medical image capabilities at or near the point of injury are often limited, rendering ultrasound the most convenient imaging platform, due to its small size, portability, and low power requirements

The most used triage examination with ultrasound imaging is the Extended Focused Assessment with Sonography in Trauma (eFAST) protocol that quickly scans the thoracic and abdominal cavities for the presence of free fluid in the peritoneal, pericardial, and pleural spaces. Air in the pleural space indicates pneumothorax, which must be treated before it worsens into a potentially fatal tension pneumothorax injury. Fluid presence in the thoracic or peritoneal cavity indicates a hemorrhagic injury either internal or from a traumatic wound to the torso that may be actively worsening. Thus, a positive diagnosis on an eFAST exam often requires immediate surgical intervention which in remote medical scenarios will require immediate medical evacuation.

However, an eFAST exam is only as valuable as the quality of the imaging and the skill level of the clinician interpreting them, therefore performing exams accurately can be exceptionally challenging for inexperienced operators during high-stress, mass-casualty situations or in remote, emergency settings. To acquire accurate information from each scan point, the transducer must be properly positioned so that relevant anatomical information is visible in the ultrasound image. Furthermore, interpretation of ultrasound images requires highly trained personnel such as a skilled radiologist, who are likely not readily available at or near the point of injury. As a result, automation strategies for improving image acquisition and interpretation are needed to make ultrasound image-based triage for the injured warfighter accessible at or near the point of injury.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings provide visual representations which will be used to describe various representative embodiments more fully and can be used by those skilled in the art to better understand the representative embodiments disclosed and their inherent advantages. In these drawings, like reference numerals identify corresponding or analogous elements.

FIG. 1 illustrates overview of eFAST scan sites, in accordance with various embodiments of the present disclosure.

FIG. 2 illustrates prediction results by Scan Point/Site for DarkNet-19, in accordance with various embodiments of the present disclosure.

FIG. 3 illustrates a diagram showing robotic arm setup for ultrasound imaging, in accordance with various embodiments of the present disclosure.

FIG. 4 illustrates an overhead view of a tissue phantom with scan points detected by the computer vision model, in accordance with various embodiments of the present disclosure.

FIGS. 5A, 5B, 5C illustrate performance guidance AI models with ground truth overlays, in accordance with various embodiments of the present disclosure.

FIG. 6 illustrates a comparison of probe adapter design and probe swap mechanism, in accordance with various embodiments of the present disclosure.

FIGS. 7A, 7B, 7C, 7D illustrate an overview of The Gear Design ultrasound probe holder, in accordance with various embodiments of the present disclosure.

FIGS. 8A, 8B, 8C, 8D, 8E, 8F illustrate an overview of the Modular Design ultrasound probe holder, in accordance with various embodiments of the present disclosure.

FIGS. 9A, 9B, 9C, 9D, 9E, 9F illustrate an overview of The Dual-End Design ultrasound probe holder, in accordance with various embodiments of the present disclosure.

FIGS. 10A, 10B, 10C, 10D illustrate test platforms for evaluating probe adapters, in accordance with various embodiments of the present disclosure.

FIG. 11 illustrates representative ultrasound images from repeatability testing with tissue phantom, in accordance with various embodiments of the present disclosure.

FIG. 12 illustrates a flowchart of different testing regimens, in accordance with various embodiments of the present disclosure.

FIGS. 13A-D illustrates force repeatability and probe swapping time results for probe adapters, in accordance with various embodiments of the present disclosure.

FIGS. 14A-D illustrates ultrasound image repeatability results for probe adapters, in accordance with various embodiments of the present disclosure.

FIG. 15 illustrates comparison of ultrasound systems on the Dual-End Probe adapter in accordance with various embodiments of the present disclosure.

FIG. 16 illustrates a system block diagram including an example of a computing device that may be used in implementing one or more features of the disclosure, in accordance with embodiments of the present disclosure.

FIG. 17 provides an overview of animal study procedures and image capture timepoints, in accordance with various embodiments of the disclosure.

FIG. 18 provides an overview of ultrasound image dataset structure and processing for images captured in swine for eFAST AI model training, in accordance with various embodiments of the disclosure.

FIG. 19 provides an overview of AI model types, in which a summary of data flow for cFAST AI model training is shown, in accordance with various embodiments of the disclosure.

FIG. 20 provides an overview of how M-mode images were generated from B-mode frames using rib guidance AI models.

FIGS. 21A, 21B illustrate an example graphical user interface, in accordance with various embodiments of the disclosure.

FIG. 22 provides an overview of an example robotic configuration for automated eFAST in swine, in accordance with various embodiments of the disclosure.

FIGS. 23A, 23B illustrate guidance AI performance for anatomical locations, in accordance with various embodiments of the disclosure.

FIGS. 24A, 24B, 24C, 24D illustrate diagnostic AI confusion matrices for each diagnostic model, in accordance with various embodiments of the disclosure.

FIGS. 25A, 25B, 25C illustrate an evaluation of a real-time eFAST application, in accordance with various embodiments of the disclosure.

FIGS. 26A, 26B, 26C, 26D illustrate performance evaluation in swine, in accordance with various embodiments of the disclosure.

FIGS. 27A, 27B illustrate RoboFAST thoracic US images, in accordance with various embodiments of the disclosure.

FIG. 28 illustrates a summary of eFAST image capture times, in accordance with various embodiments of the disclosure.

### DETAILED DESCRIPTION

Medical imaging-based triage is important for emergency medicine in both civilian and military settings. Ultrasound imaging can be used to rapidly identify free fluid in abdominal and thoracic cavities which could necessitate immediate surgical intervention. One specific application is the eFAST, where pneumothorax, hemothorax, or abdominal hemorrhage injuries are identified. However, the diagnostic accuracy of an eFAST exam depends on obtaining proper scans and making quick interpretation decisions to evacuate casualties or administer necessary interventions. Proper ultrasound image capture requires a skilled ultrasonography technician who is likely unavailable at the point of injury where resources are limited.

Ultrasound imaging is an important tool in emergency medicine for initial assessment of injuries and triaging patient status for prioritizing medical evacuation resources. The utility for ultrasound imaging can be extended if the skill threshold can be lowered for acquisition and interpretation of scan results so that imaging-based triage can be more common in the pre-hospital military or civilian setting. The cFAST triage application described herein is very useful for detecting free fluid in the thoracic or abdominal cavities and positive eFAST diagnosis can often require urgent surgical intervention. AI image interpretation models for identifying positive and negatives status at each scan point can streamline this process without needing ultrasonography expertise if high accuracy AI models can be trained for this application. To this end, different approaches and model architectures were highlighted for each eFAST scan point to optimize their performance.

In at least one embodiment, the source of the ultrasound images to be analyzed is immaterial and may be from manual scans or robotic/automated scans of the patient. The AI model as explained herein was trained on a variety of ultrasound images reflecting a variety of injuries and healthy examples. The model performs its analysis on a processor connected to a memory and the source of the ultrasound images. The model is configured to perform the analysis associated with an eFAST examination by reviewing the ultrasound images for the presence of fluid (e.g., air pocket or loose fluid internal to the patient). In at least one embodiment, different AI models are used for different scans used as part of the cFAST analysis. The model is trained on a variety of ultrasound images reflecting the conditions to be found if present by an eFAST examination. The following discussion explains the development of the model and ways that one or more of those models have been improved. As part of the embodiments of this disclosure, the model can review ultrasound images from both uninjured and injured patients and determine if the patient does in fact have the internal injuries detectable by eFAST to assist in the triage in a mass casualty situation.

To improve ultrasound interpretation, as described herein AI models were developed to identify key anatomical structures at cFAST scan sites, simplifying image acquisition by assisting with proper probe placement. These models plus image interpretation diagnostic models may be paired with two real-time eFAST implementations. An implementation described herein is the use of these AI models with a robotic imaging platform capable of providing semi-autonomous image acquisition combined with diagnostic image interpretation. Another implementation is a manual AI-driven ultrasound cFAST tool that uses guidance models to select correct frames prior to making any diagnostic predictions. Both real-time approaches were used in a swine injury model, for example, and performances highlighted in an emergency medicine application. As described herein, AI can be deployed in real time to provide rapid triage decisions, lowering the skill threshold for ultrasound imaging at or near the point of injury.

Having diagnostic models to interpret medical images, however, only addresses part of the challenge with performing eFAST exams. The other issue is adequate medical image acquisition for discernable image capture so that AI models can interpret the presence of injury. For this, the trained deep learning models described herein are integrated with automated cFAST image acquisition and interpretation in real-time, allowing for model inferencing in live and euthanized swine. AI and robotics are applied to the eFAST exam, utilizing computer vision AI to guide a robotic platform to the relevant scan points of the cFAST exam. Another acquisition method may include a handheld AI-driven US application that guides the user to the correct scan site using AI guidance models and then runs AI diagnostic models, for example. Having a trained model to classify diagnostics for ultrasound images can also objectify the process of making decision during triage and lower the skill threshold needed to obtain diagnosis predictions once ultrasound images are acquired.

The development of artificial intelligence (AI) has accelerated in several fields of technology, including the healthcare industry. In the medical imaging field, AI has improved efforts in patient care and medical diagnoses of disease and abnormalities. AI not only reduces the time it takes to diagnose these problems, but also gives supplemental insight to medical providers by finding and interpreting abnormalities that could have otherwise been missed by a human eye unfamiliar with discerning nuanced features. In addition, technological advancements have allowed for improved care administration for trauma patients on the battlefield. One example is the use of internet-based video communication to receive real-time advice from medical professionals to properly treat or address casualty patients. Closed-loop systems for fluid or drug administration utilize fully automated medical administration approaches to stabilize patients that are being transported to more definitive care. Robotics improve the treatment administration of surgical interventions through telerobotic platforms.

Robotics and computer vision technology can simplify image acquisition. As a first step towards this larger goal, prototypes for ultrasound probe securement using a robotics platform were developed. The ability of four probe adapter technologies to precisely capture images at anatomical locations, repeatedly, and with different ultrasound transducer types were evaluated across more than five scoring criteria. Testing demonstrated two of the adapters outperformed the traditional robot gripper and manual image capture, with a compact, rotating design compatible with wireless imaging technology being most suitable for use at the point of injury. The best prototypes then were integrated into a robotic platform with computer vision and deep learning image interpretation models to automate image capture based on anatomical landmarks. The result is that a lower level of skill will be needed for medical imaging-based triage, enabling this procedure to be available at or near the point of injury.

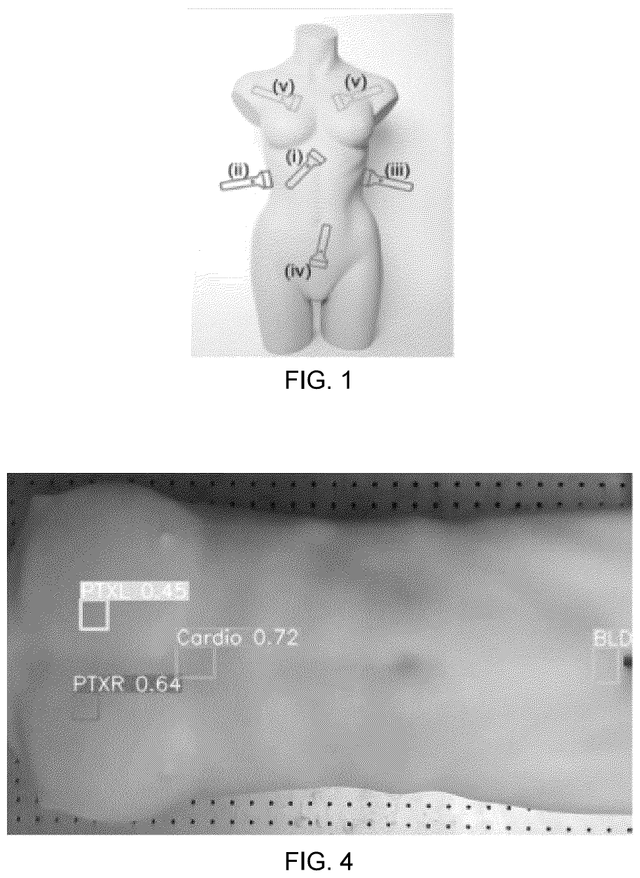

Diagnosing internal injuries in combat casualties can be very challenging. The eFAST exam uses ultrasound imaging to scan the thorax and abdomen for free fluid for injury diagnosis. Typical locations for eFAST scan sites are illustrated in FIG. 1, which illustrates overview of eFAST scan sites. Views include (i) subxiphoid, (ii) right upper quadrant, (iii) left upper quadrant, (iv) pelvic, and (v) intercostal scan points, in accordance with various embodiments of the present disclosure.

However, ultrasound image capture requires a skilled and experienced user who is likely unavailable in casualty care scenarios where trained personnel are limited. Therefore, automating these procedures may improve trauma patient outcomes.

Ultrasound imaging is an essential tool for emergency triage to allocate limited resources to severely injured casualties especially during remote medical situations such as those faced in combat casualty care. This need is expected to rise in the future, especially where contested airspace make medical evacuations even more limited. Unfortunately, ultrasound imaging is only a viable triage tool if proper images can be captured, which is not guaranteed in high stress situations or by non-subject matter expert ultrasound sonographers. The robotic platform provided herein allows for the automated image acquisition for reliable ultrasound image collection.

In at least one embodiment, imaging automation utilizes robotic systems to hold the transducer while capturing ultrasound images. At a lower automation level, the robot can be remotely controlled for capturing images without the operator needing to be at the scan site. This has the benefit of allowing a skilled operator to continue image acquisition, but it may still require human input throughout the entire scan. Furthermore, latency delays become a challenge as the distance between the operator and robot expands. This is further complicated in remote emergency medicine situations where remote communication may not be feasible. Remote robotic imaging has been used for obstetric, cardiovascular, and abdominal imaging applications. At a higher automation level, progress has been made toward fully autonomous image capture through the integration of mechanisms to account for force applied to the subject, whether it be through force sensors or spring mechanisms to regulate application force. These mechanisms have been successfully integrated with different robotic platforms to ensure no harm is done to the subject during scanning and that sufficient force is applied for ultrasound image capture. The next step in automated image capture is generating robotic movements to properly capture images. This may be done offline by reference tissue phantoms or a camera attached to the robot allowing for an operator to precisely pinpoint where the robot should move and in what order to properly collect images. This can be done in real-time instead, using computer vision AI to identify scan points based on patient anatomical landmarks. However, computer vision alone cannot acquire proper ultrasound images due to the need for precise probe angling and positioning. Instead, more advanced ultrasound visual imaging techniques are needed which utilize ultrasound image feedback to position the robot properly for image capture. Approaches have used reinforcement learning to try and learn how an experienced sonographer captures images as well as convolutional neural networks for assessing image stability or object tracking.

For designing a robotic system for eFAST assessment, a few additional challenges must be considered. First, as this is an emergency medicine triage tool, speed of the examination is critical. An average eFAST exam can take approximately 4 minutes, so robotic image capture must be comparable to this. This will require more rapid decision making by the image capture setup than may be required for many other automation applications being considered. Second, the thoracic scan points traditionally use a linear ultrasound probe, while abdominal scan points use a curvilinear probe type, requiring the imaging system to be able to swap probes automatically while maintaining image capture accuracy.

As a first step toward automating eFAST image acquisition, focus was on methods for securing an ultrasound probe to a robotic imaging platform. Purpose-built adapters are needed for accommodating the unique design considerations specific to eFAST image capture. Specifically, in accordance with certain embodiments, four different ultrasound probe holder adapters paired with a Universal Robot arm were evaluated to determine if the platform can be configured optimally for rapid, accurate, and versatile ultrasound image capture that conducting an eFAST examination will necessitate.

A robotic platform with adaptations for ultrasound probe attachment, integrating computer vision (CV) and real-time artificial intelligence (AI) guidance technology, provides an alternative approach to manual image acquisition. While CV alone may enable navigation to the approximate scan sites of the body for imaging, AI models provide real-time image-based feedback which further guides robotic navigation, ensuring acquisition of anatomical views needed for image interpretation. To improve ultrasound interpretation, AI models were developed to identify key anatomical structures at cFAST scan sites simplifying image acquisition by assisting with proper probe placement. These models plus image interpretation diagnostic models may be paired with various real-time cFAST implementations. A robotic platform for automating an eFAST exam suitable for simplifying triage on the future battlefield and other remote medical locations is described herein. More particularly, these AI models are used with a robotic imaging platform capable of providing semi-autonomous image acquisition combined with diagnostic image interpretation. As described herein, AI can be deployed in real time to provide rapid triage decisions, lowering the skill threshold for ultrasound imaging at or near the point of injury.

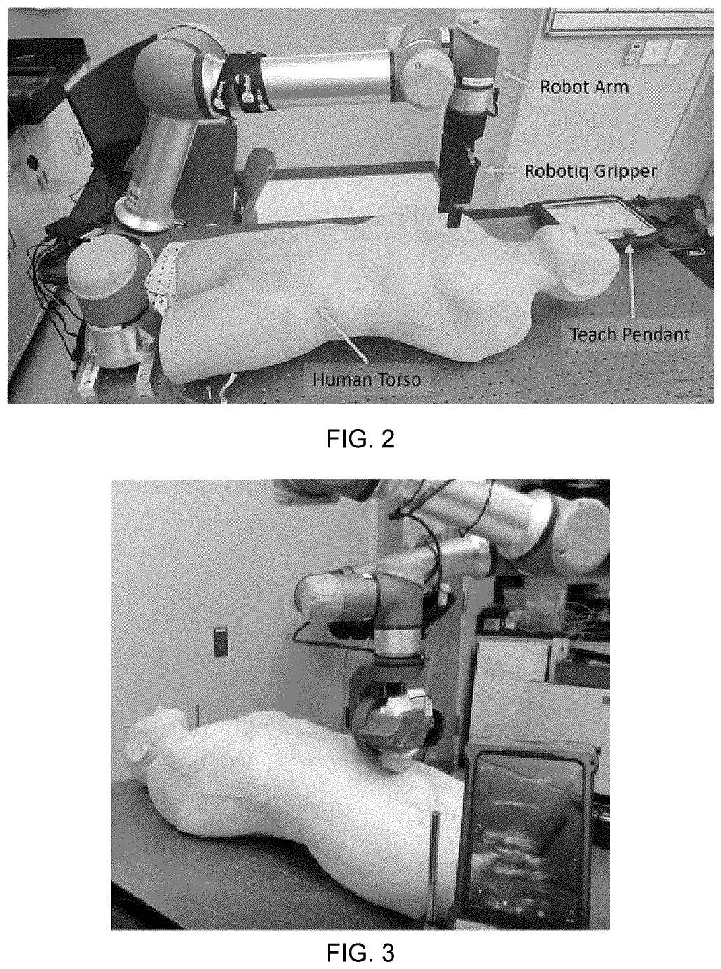

In at least one embodiment illustrated in FIGS. 2-3, there is a system having at least one ultrasound transducer (e.g., linear or curvilinear probe), a robotic arm (e.g., UR5c system (Universal Robots, Odense, Denmark)) configured to pass over a patient, an adapter configured to hold the ultrasound transducer and press the ultrasound transducer against the patient, an optional camera configured to image the outside of the patient, an optional pressure sensor attached to the transducer and configured to detect the amount of pressure being applied by the ultrasound transducer, a controller having a model to determine the location of the ultrasound transducer relative to a measurement area (or scan site) on the patient, and an optional display. Alternatively, the pressure sensor is replaced or augmented by a spring mechanism in the adapter to regulate application force of the ultrasound transducer against the patient's skin. In a further embodiment, the system includes a gel dispenser (or a gel dispensing system) that includes a peristaltic pump with tubing routed to the ultrasound probe head and contains a custom printed gel distribution attachment for effective gel delivery of the tissue surface. In a further or additional embodiment, the probe adapter is configured to hold both a linear probe and a curvilinear probe that are switchable between them by the robotic arm (see, e.g., Dual-end design).

As discussed later, there were three adapters tested versus a commercially available adapter to determine the best adapter for use in the system. In at least one embodiment, the controller is software running on a processor. In at least one embodiment, the model is configured to detect anatomical landmarks that are present in the ultrasound image and possibly the image originating from a camera and then to direct movement of the robotic arm to place the ultrasound transducer on the patient in the measurement zone to perform an ultrasound image capture for use in diagnosing the patient (e.g., cFAST examination). The pressure sensor provides a signal to the controller to allow for a determination to be made if a sufficient level of pressure is being applied, too little pressure is being applied, or too much pressure is being applied by the robotic arm to allow for adjustment by the robotic arm. The display may show the ultrasound scan or other system information and/or provide an interface with the system.

In at least one embodiment during testing, the system includes a robotic platform integrated with CV technology for locating scan points on a phantom subject. Ultrasound probe adapter designs were integrated with the end of the robotic arm, enabling ultrasound image acquisition. Furthermore, guidance AI models were developed that allow the robotic arm to capture images with relevant anatomical features.

Robot navigation begins with a computer vision algorithm that estimates ultrasound scan points based on anatomical features (or landmarks). These estimated scan points are programmed as robot scan destinations which the robot positions itself to, followed by adjusting the ultrasound approach angle and applying tissue compression until a force threshold is reached. At this point, object detection AI models trained for identifying ribs, kidney, or bladder objects are used to evaluate captured ultrasound images for proper scan site orientation. For initial development, the robot continued to scan the tissue site in a grid fashion until the AI model determines the robot is at the proper scan location. In its present form and for future development, the AI model communicates location of objects in the image and the robot will move or rotate the ultrasound probe accordingly to allow for relevant regions of interest to be centered in the image.

Once the robot has been guided to the proper scan location, diagnostic images are captured by either remaining static for collecting an m-mode image or by adjusting the angle while fanning the ultrasound probe to capture a range of angles for ensuring the injury is identifiable. Diagnostic AI models then can create predictions based on these captured images for automating interpretation in real time.

Ultrasound probe adapters were designed and integrated with the end of the Universal Robot (UR5c) arm (Universal Robots, Odense, Denmark), enabling the UR5e to apply an US probe to a subject and acquire ultrasound images. Reference FIG. 3 which illustrates a diagram showing robotic arm setup for ultrasound imaging. The UR5e (‘Robot Arm’) is table mounted and positioned over a torso phantom (‘Human Torso’) and equipped with an end-adapter (Robotiq Gripper is shown). Robotic motion control is programmed via the Teach Pendant, in accordance with various embodiments of the present disclosure. The UR5e was equipped with a depth-sensing camera for use with CV technology developed to identify scan points on a phantom subject (FIG. 4). Furthermore, guidance AI models were developed which enhanced image acquisition through real-time ultrasound image feedback. This allowed the UR5e to capture ultrasound images with relevant anatomical features in view to allow for diagnostic injury interpretation, towards advancements in triage evaluation

CV models (YOLOv8) were trained to identify eFAST scan points of a tissue phantom (FIG. 4). FIG. 4 illustrates an overhead view of the tissue phantom taken by an Intel RealSense camera integrated with the end of the UR5e robotic arm. Scan points detected by the computer vision model are shown, as indicated by the colored bounding boxes, in accordance with various embodiments of the present disclosure. Identification of scan points allowed the UR5e to autonomously navigate to each scan point and apply an ultrasound probe to the tissue for ultrasound imaging as illustrated in FIG. 3.

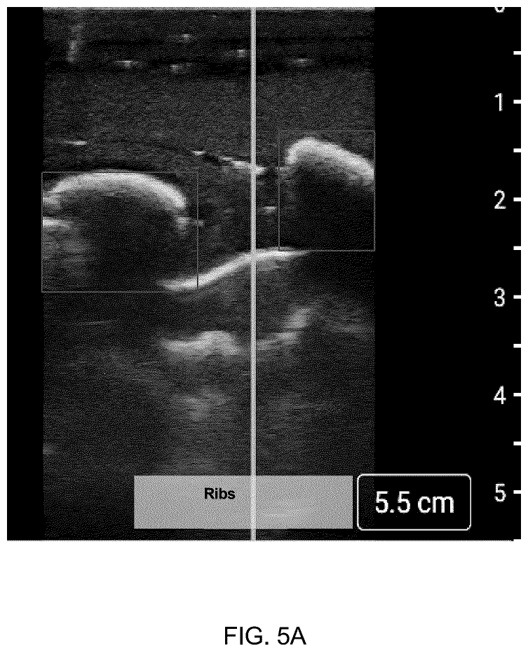

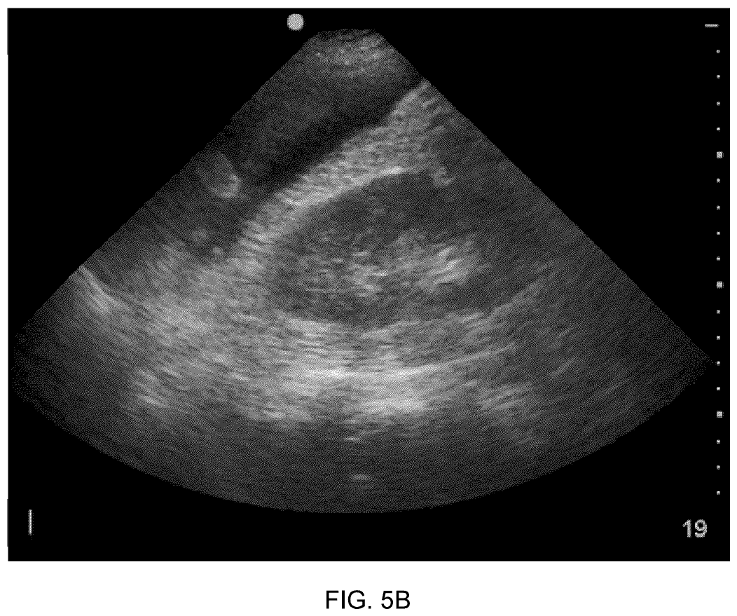

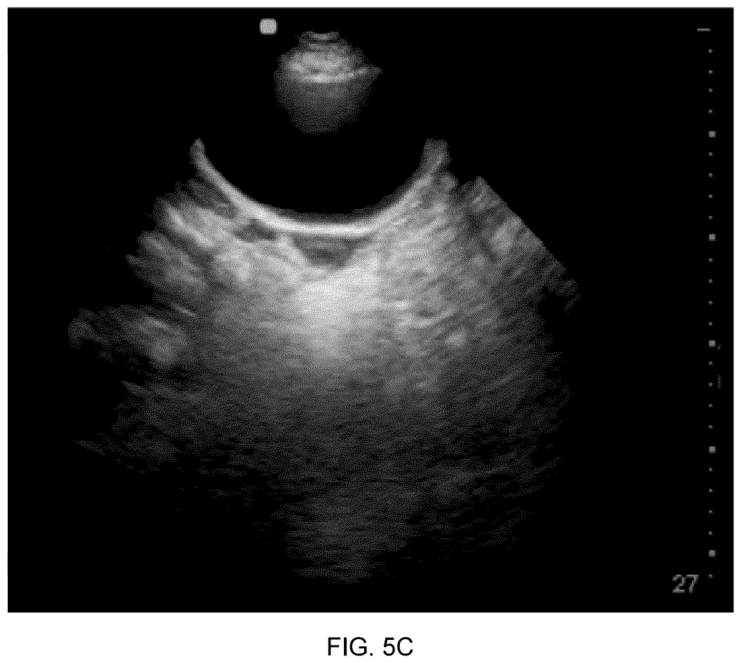

The probe adapters were designed, prototyped, and tested with the robot. A single adapter, designed for use with a dual-ended wireless ultrasound probe model, was selected through testing. Additionally, CV models were successful at sufficient accuracy across each eFAST scan point. Similarly, guidance AI models for the ribs, kidneys, and bladder were trained on swine anatomy images with 0.72, 0.48, and 0.64 intersection over union scores compared to ground truth labels, respectively. FIGS. 5A-5C illustrate performance of the guidance AI models for ribs, kidneys, and bladder with ground truth overlays overlaid in red, in accordance with various embodiments of the present disclosure. FIG. 5A, for instance, uses red bounding boxes in connection with ground truth overlays for ribs. The AI models provide feedback to the UR5e to allow it to continue strategically scanning an area until relevant underlying structures which are necessary for diagnostic interpretation are identified by the AI model.

The system utilized a custom 3D printed probe holder attachment capable of connecting to different ultrasound probe types and allowing for easy swapping between probe types. The top design developed uses a wireless ultrasound system and a rotating mechanism that the robot can manipulate to flip the probe 180 degrees, as is needed to swap between linear and curvilinear probe heads on the ultrasound system. To effectively capture images, a gel dispensing system was developed. This includes a peristaltic pump with tubing routed to the ultrasound probe head and contains a custom printed gel distribution attachment for effective gel delivery of the tissue surface.

### Ultrasound Probe Adapters

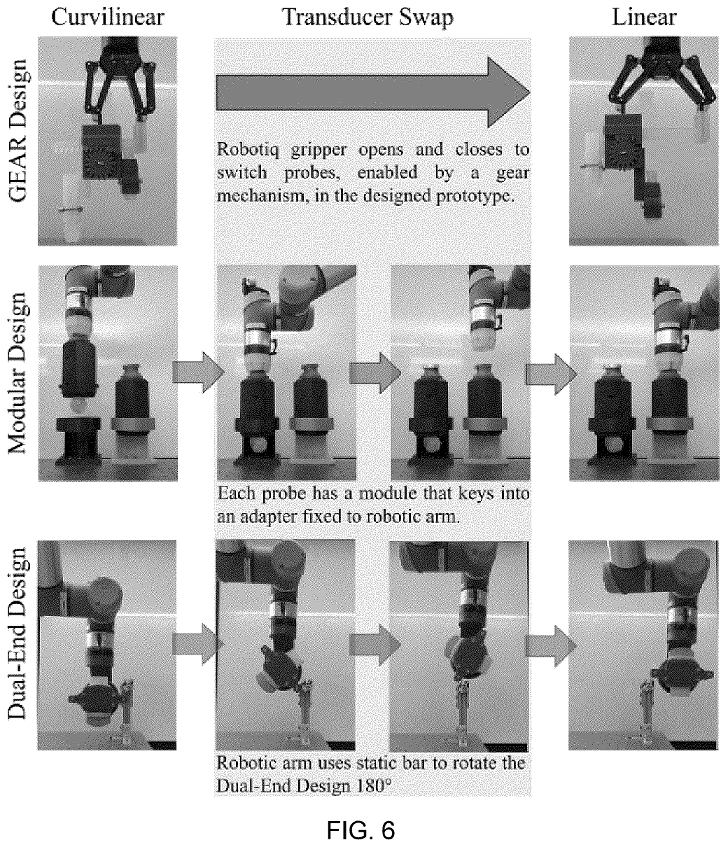

A Universal Robot 5e (UR5e) was used for attachment of various probe holder designs and movement of an ultrasound probe to acquire proper images (FIG. 6). The UR5c system has a maximum payload of 5 kg and 6 degrees of motion freedom, allowing for image capture at each of the various eFAST scan points. UR5e motion is programmed using on-board control capabilities via the Teach Pendant; reference FIG. 2 which illustrates prediction results by Scan Point/Site for DarkNet-19, in accordance with various embodiments of the present disclosure. Each probe adapter design (discussed later) integrates with the UR5c, allowing the robot to hold an ultrasound transducer for performing the cFAST exam. A program was developed which provided UR5e control over the positioning of the probe using force and or obtained through an integrated sensor (Robotiq, Lévis, Quebec, Canada).

Probe adapters were developed with several key design constraints. The adapters needed to ensure contact between the ultrasound probes and the subject (or patient), considering the range of motion of the robotic arm and other limitations such as length of the cord for standard ultrasound systems. Therefore, compact designs were prioritized. The probe adapter prototypes also required the ability to change between at least two transducers that are commonly needed to complete an eFAST exam-linear and curvilinear probes. The higher-frequency linear probe is used for thoracic imaging as the key structures are shallower while the lower-frequency curvilinear probe allows for visualizing deeper abdominal organs. This design feature would allow for the appropriate probe to be selected as necessary for each specific scan site. Therefore, modular and self-contained designs which addressed this need for probe exchange were considered as well. Additionally, considering the robotic platform application of medical imaging-triage, designs needed to account for reliability and time of probe exchange to decrease the time needed to perform the eFAST procedure. Since navigation to each scan point would be controlled by the robotic arm, duration of the exam would be most affected by the time required to swap probes. Finally, the probe adapter designs needed to apply force to the subject in a reliable manner which would allow for clear image capture at multiple scan locations. Therefore, designs needed to securely hold the probe in place under repeatedly applied forces.

Four different ultrasound probe holders were evaluated based on these design criteria. Three purpose-built prototype adapters were compared against a fourth commercially available gripper, the 2F-140 adaptive gripper (Robotiq, Lévis, Quebec, Canada). FIGS. 6-9 illustrate the three prototype adaptors. Using this end-adapter's adjustable grip width, it could be manipulated to hold any ultrasound probe for testing

Each custom probe adapter incorporated a probe swapping mechanism for case of performing a full cFAST exam. The probe adapters were designed to hold ultrasound probe models, for example a Terason ultrasound probe (Terason, Burlington, MA, USA). One probe adapter was fitted around a dual-sided wireless ultrasound probe, Vscan Air™ CL, (GE HealthCare Technologies, Inc., Chicago, IL, USA). However, to avoid damaging ultrasound probes during preliminary testing of the robotic imaging platform, a mock ultrasound probe of identical shape and size was cast with polyurethane (Clear Flex 50, Smooth-On, Easton, PA, USA) after an inverse mold was made using silicone rubber (Eco Flex 00-31 Near Clear, Smooth-On, Easton, PA, USA). In addition, each probe adapter utilized a mold of the precise geometrical shape around the ultrasound probe using an epoxy resin (Smooth-On, Easton, PA, USA) so that the probe adapters could hold a more regular, defined geometry.

The three prototype adapters include the Gear Design, the Modular Design, and the Dual-end Design illustrated in FIGS. 6-9.

FIG. 6 illustrates a comparison of each probe adapter design and probe swap mechanism, in accordance with various embodiments of the present disclosure. Three different designs are shown: Gear design (Row 1), Modular Design (Row 2), and Dual-End Design (Row 3). For each, the mechanism for probe swapping is shown from curvilinear (left) to linear (right) transducer. Intermediate images are shown when necessary to better display the probe swap technique.

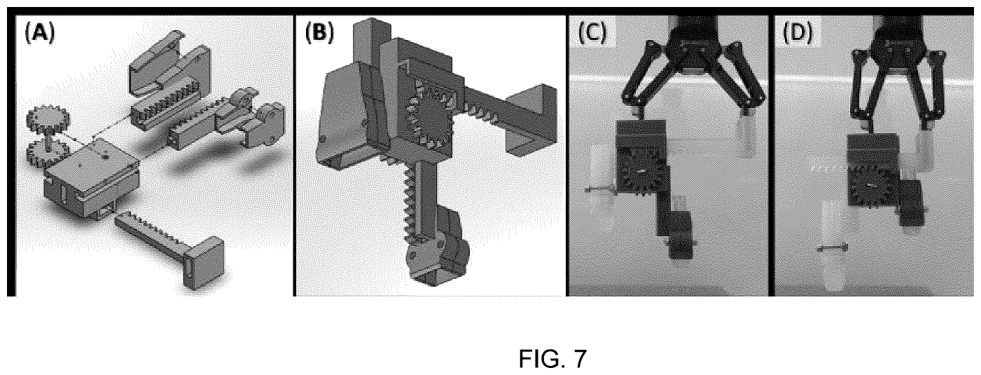

The Gear Design was devised as a robotic arm attachment specifically designed for seamless ultrasound probe transitions that significantly reduce the amount of time required to switch probes. It was engineered with a triple rack and dual pinion system (FIGS. 7A-7D) that ensures efficient and synchronized movements of both ultrasound probes (FIG. 6, Row 1). When one probe is elevated into its operational position, the other is simultaneously and smoothly retracted into a standby position (FIG. 6, Row 1).

With regard to FIGS. 7A, 7B, 7C and 7D, an overview of The Gear Design ultrasound probe holder is illustrated. An engineering diagram of The Gear Design in (FIG. 7A) exploded view and (FIG. 7B) assembled view. The design can switch between linear (FIG. 7C) and curvilinear (FIG. 7D) transducer configurations, in accordance with various embodiments of the present disclosure.

The device was attached to the 2F-140 adaptive gripper, which was used to control the third rack and pinion. The opening and closing motion of the gripper slides the rack left or right while rotating the pinion clockwise or counterclockwise. The rotation of this pinion controls the rotation of the pinion attached on the opposite side. This controls the vertical motion of the two probe-holding racks. Therefore, probes are exchanged by opening or closing the gripper. The operations orchestrated by the subtle actions of the gripper eliminate the need for manual interchange and reduce the time required for transition. The device was manufactured with resin (Formlabs, Somerville, MA, USA) by additive manufacturing.

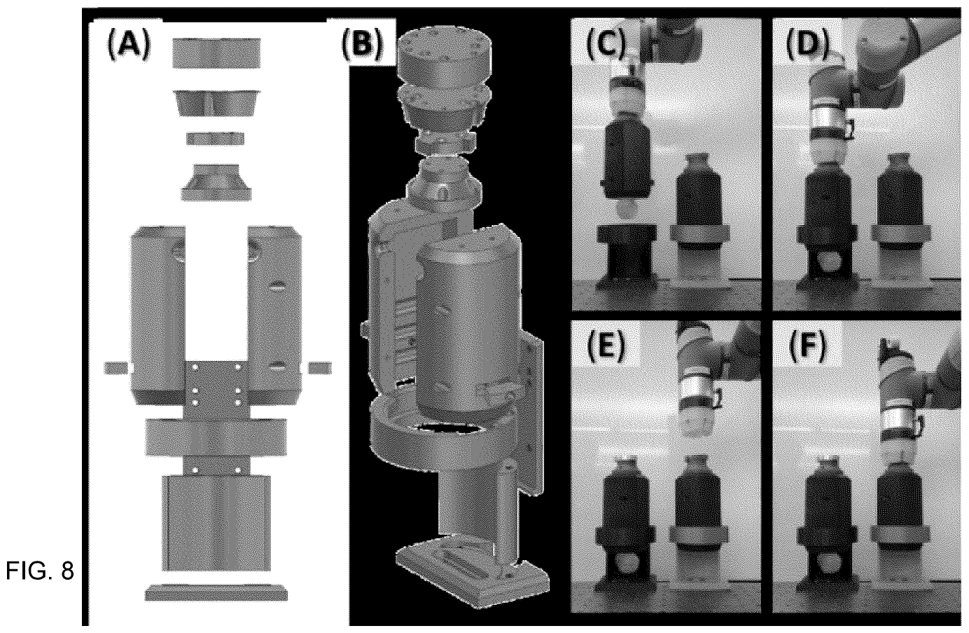

The Modular Design can swap ultrasound probes through rapid docking and undocking of each probe. The prototype was fabricated with PLA (Raise3D, Irvine, CA, USA) and resin (Formlabs, Somerville, MA, USA) by additive manufacturing. Each probe was secured within a modular enclosure with a design key at the top surface of the device as well as a design key on either side of the device (FIG. 8). This modular probe adapter was then positioned within a docking station affixed to the operational surface.

With reference to FIGS. 8A, 8B, 8C, 8D, 8E, 8F, an overview of the Modular Design ultrasound probe holder is illustrated, in accordance with various embodiments of the present disclosure. An engineering diagram of the Modular Design front view in FIG. 8A and an oblique exploded view in FIG. 8B are shown. FIGS. 8C-8F illustrate that the design can swap between linear and curvilinear transducer housed in docking stations.

For procedure initialization, the receiving end part was integrated with the robotic arm allowing for the robotic arm to lower down and receive the top keyed end of the modular probe adapter. The robotic arm fitted with the end receiver then rotates around the keyed module, held stationary by the docking station, locking the modular adapter into place. At this stage, the robotic arm can lift the module with the probe

For swapping between transducers, the module is lowered to the docking station by the robotic arm (FIG. 6, Row 2), lining up the side keys with the slots of the dock. The robotic arm's twist motion is then reversed until the module's side keys are locked into the docking station slots. Continued rotation releases the module's top key from the robotic arm's end receiver. Once released, the robotic arm is detached from the probe module. The robotic arm can then perform the reverse operation for attaching to a different transducer module (FIG. 6, Row 2).

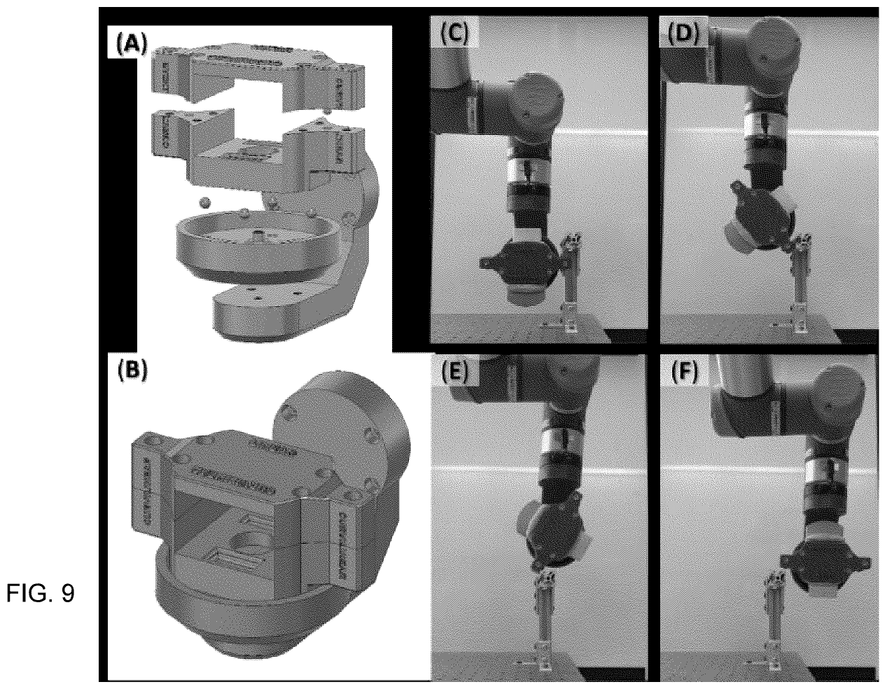

The Dual-End Design was engineered for probe models with scanning functionality available on either end of the transducer. In its current form, the design used the Vscan Air™ CL, a commercially available wireless dual ended model featuring Wi-Fi and Bluetooth capabilities, with linear and curvilinear probes on each end. The model's wireless feature overcomes the limitation imposed by traditional wired models which potentially limit the robotic arm's range of motion during operation. The probe adaptor was designed to rotate the ultrasound probe in place and lock at two set positions for the curvilinear or linear end of the probe (FIG. 9). The robotic arm was programmed to exchange probe ends by rotating the probe adapter 180° around a static bar. This programmed method of exchange bypasses the need for additional motorized features and makes use of the probe's wireless benefits (FIG. 6, Row 3). While rotating from its starting position, four spring-loaded steel balls recess into the holder and allow the Vscan Air™ to find its next set position. When the holder reaches one of the endpoint positions, the spring-loaded balls will home into its divot, and lock the ultrasound probe in place. The prototype was fabricated with PLA (Raise3D, Irvine, CA, USA) and resin (Formlabs, Somerville, MA, USA) by additive manufacturing.

With regard to FIGS. 9A, 9B, 9C, 9D, 9E, and 9F, an overview of the Dual-End Design ultrasound probe holder is illustrated. Engineering diagrams of The Dual-End Design in exploded view (FIG. 9A) and assembled view (FIG. 9B) are shown. FIGS. 9C-9F illustrate that the design can swap between linear and curvilinear transducer by rotation around a horizontal bar, in accordance with various embodiments of the present disclosure.

### Testing of Probe Adapters

Two different testing configurations were used to evaluate the performance of the varied probe holder designs. The eFAST testing configuration used a torso mannequin which allowed the ultrasound probe to be positioned at the proper eFAST scan sites. However, this testing setup was not ultrasound compliant, so a second testing setup was configured which allowed the robotic arm to capture ultrasound images of the femoral artery region of an ultrasound compliant tissue phantom. These two testing modalities are detailed below.

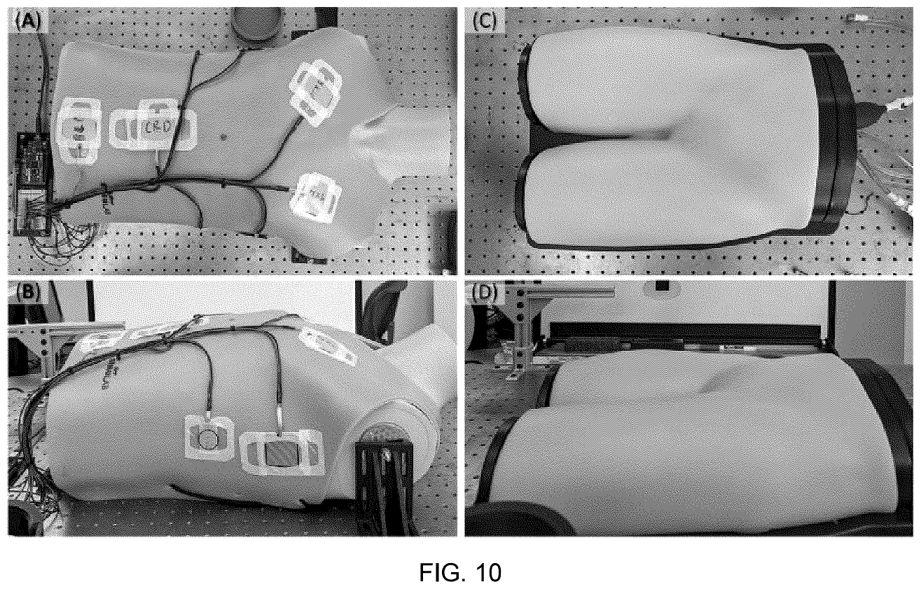

A torso mannequin (Amazon, Seattle, WA, USA) was fitted with a silicone cFAST vest (Simulab, Seattle, WA) situated with force sensing resistors (SparkFun Electronics, Niwot, CO, USA) approximately placed at each of the eFAST scan points (FIGS. 10A, 10B). For each probe adapter design, the robot was programmed to complete an eFAST exam on the torso mannequin. Robotic arm movements were controlled by specifying the coordinates for the desired position of the robotic arm, which would place the probe at the desired contact site of the body. The robotic arm was then programmed to continue moving towards the body until a collision was detected and contact was made. The variation in geometry of each design required slight variations in the path of travel; however, each probe adapter was programmed to achieve contact with the body and apply force at each scan point. Each test was conducted five times (n=5) with both the curvilinear and linear probes for a total of ten trials conducted per each of the four probe adapter approaches.

Referring now to FIGS. 10A, 10B, 10C, and 10D, test platforms for evaluating probe adapters are shown, in accordance with various embodiments of the present disclosure. Torso mannequin with force sensing resistor configuration for testing repeatability of full cFAST exam. FIG. 10A shows a top view and while FIG. 10B shows a side view Ult issue phantom of femoral region used for image repeatability testing. FIG. 10C shows a top view and FIG. 10D shows a side view.

Each test began with the probe adapter and the ultrasound probe attached to the robotic arm. Through programmed robotic movements, the probe scanned each cFAST scan point, and repeated successful contact with the scan site was determined by force sensing resistors placed at each scan site. Before and after the cFAST scan, the UR5e was programmed to have the ultrasound probe make contact with a force sensor (Mark-10, Copiague, NY, USA). The UR5e was programmed to repeat each scan with precise positions specified so that any difference in force measurements by the force sensor provided an indication of the repeatability of the application of force by each adapter design. Any change in the position or orientation of the probe within the adapter would alter the applied force. Therefore, the repeatability of force application provided a metric for evaluating the stability or security of the probe within the adapter. Finally, probes were exchanged at the end of each test to evaluate reliability of the probe swapping capability of each design. Additional tests (n=5) were conducted for each design to determine the time required to swap probes alone.

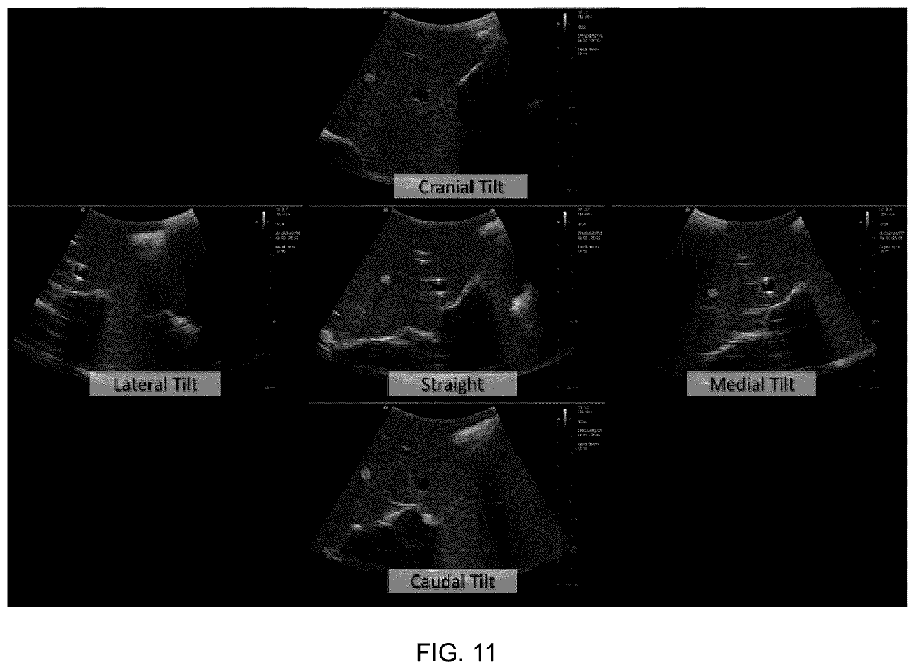

Additional tests were conducted using a Femoral Vascular Access Training Model (FIGS. 10C, 10D) (CAE Healthcare, Montreal, Canada) to evaluate the repeatability of image capture by the robotic system. This model was used as it is an ultrasound compliant material with a vasculature that allows for assessing ultrasound image quality at different angles and applied forces. In the first round of testing, the UR5e robot was programmed to scan the leg phantom using a curvilinear probe orthogonally to the surface of the right leg using the three probe adapter designs and the Robotiq adaptive gripper. For comparison, images were also captured manually by a single human operator. The probe was then tilted±20° in four directions: cranial-caudal and medial-lateral (FIG. 11). For each scanning technique (robotic and manual) ultrasound images of the phantom leg were captured using the Terason ultrasound machine, and additional images were captured by the Vscan Air™ wireless ultrasound device with the Dual-End Design. The simulated vein, artery, and/or nerve features remained in view with each of the probe orientations and scanning approaches, to be able to track anatomical features across trials for evaluating image capture repeatability. Analysis was performed using MATLAB 2022b Image Labeler toolbox by pixel labeling. Each ultrasound image was labeled by three subjects to reduce labeling bias. The x and y centroid coordinates of each image label were calculated and compared across each design to evaluate the scanning repeatability of each design.

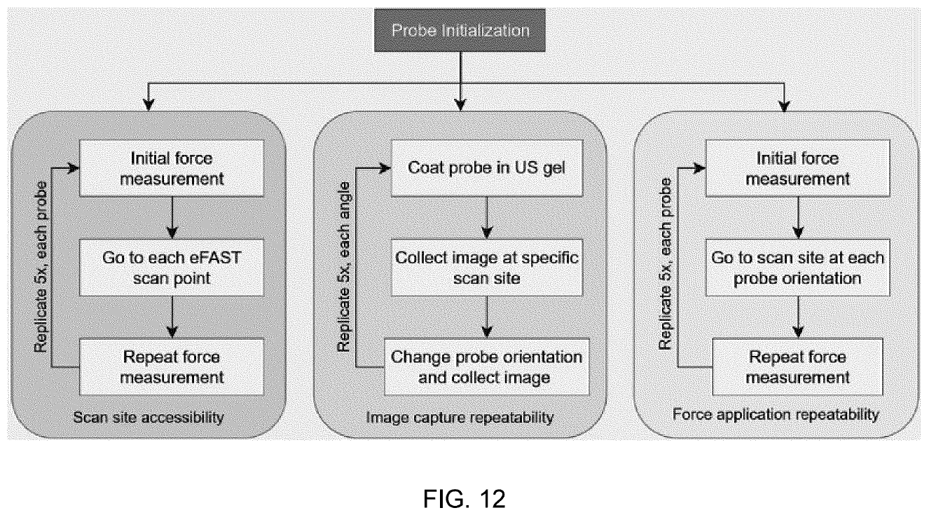

Additional tests (n=5) were performed without image capture to evaluate force application repeatability using a force sensor (FIG. 12, Right) (Mark-10, Copiague, NY, USA). In these tests, the curvilinear probe was also exchanged for the linear probe to further assess reliability of probe exchange by each design.

Referring now to FIG. 12, a flowchart of different testing regimens (from left to right) of scan site accessibility was determined with the eFAST torso model, in accordance with various embodiments of the present disclosure. Testing based on contact with force sensors at each scan site; image capture repeatability was measured with ultrasound compliant femoral region phantom and measured the variability in image capture by each design; force application repeatability was quantified before and after enough force was applied to ac e ultrasound images at different angles to assess if the ultrasound probe was properly secured in each probe adapter design.

All statistical analyses were performed with GraphPad Prism 10.1.2 (La Jolla, CA, USA). To evaluate significant differences between the different ultrasound probe adapters, one-way analysis of variance (ANOVA) models was used. This was done for force repeatability and probe swap times to compare the four probe adapters. For the image capture repeatability analysis, a repeated measures ANOVA analysis was used where the five different probe positions were treated as repeated measures for each probe adapter as well as manual image acquisition. Each of these used a post-hoc Tukey's test to measure significance wherein the significance threshold was taken as p=0.05 for all analyses. ANOVA post-hoc Šídák test was used when comparing specific groups such as evaluating the effect of ultrasound device (VScan Air™ or Terason) on image repeatability. Further, outlier exclusion criteria were used to remove data points using the ROUT method (robust regression followed by outlier identification) with a false discovery rate set at 1%.

The eFAST torso test setup allowed for assessment of each probe adapter's performance across each scan point as well as swapping between each ultrasound probe type. Starting with overall success at swapping probes and reaching scan points, all probe adapter designs were able to initialize and swap ultrasound transducers with a 100% success rate and access the scan points on the torso and phantom models with a similar high success rate. Next, the variability of applied force by each adapter design was evaluated, as the probe swapping required for an eFAST scan could result in probe placement error.

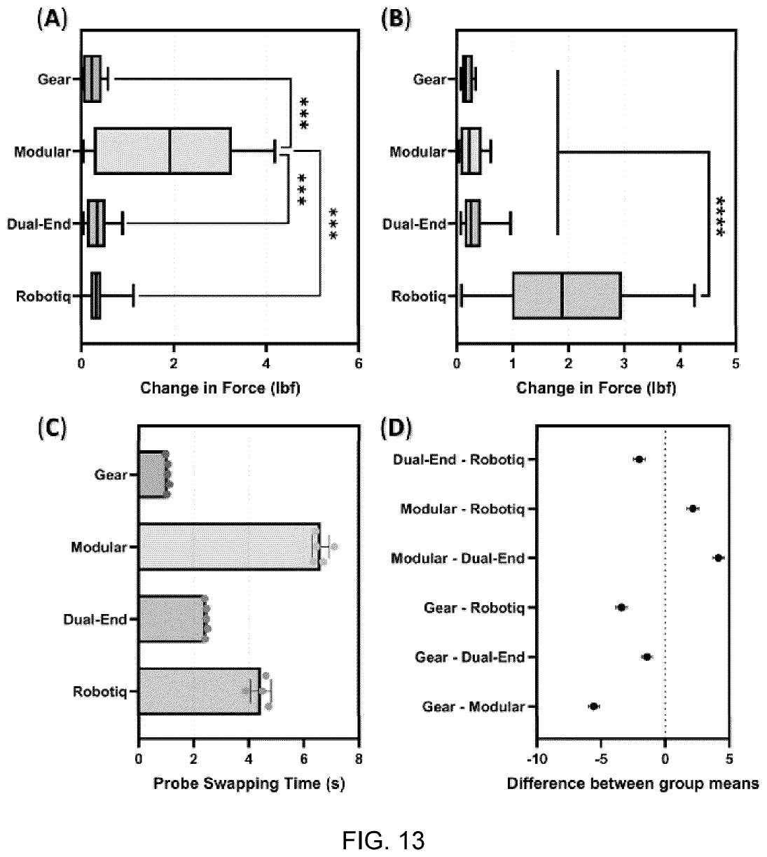

Referring now to FIGS. 13A, 13B, 13C, and 13D, force repeatability and probe swapping time results for each probe adapter are illustrated, in accordance with various embodiments of the present disclosure. FIG. 13A and FIG. 13B illustrate the absolute difference between repeated force measurement for each probe adapter (n=10) for the (FIG. 13A) torso and (FIG. 13B) phantom model. Statistically significant differences between two groups are denoted by asterisk. Box and whisker plots show the mean value and minimum and maximum values as the error bars. FIG. 13C illustrates the time needed to swap probes between curvilinear and linear (n=5). Error bars denote standard deviation. FIG. 13D illustrates 95% confidence each swap time group mean. As shown by confidence intervals not crossing ns between each probe adapter were statistically significant.

For the torso mannequin tests, the applied force of each probe adapter design was repeatable for all but the Modular Design, with each other design having a significant difference vs. the Modular Design (FIG. 13A). Further, the variability for the Modular Design was much higher compared to the other probe adapters. Conversely, for the phantom testing, the Robotiq gripper had the highest force measurement error, with each other probe design having a significant difference compared to the Robotiq gripper (FIG. 13B).

The last metric evaluated for the torso phantom test was the length of time to swap probes. The Gear Design required 1.1 seconds compared to 2.4 seconds for the Dual-End Design and 6.6 seconds for the Modular Design (FIG. 13C). Meanwhile, manual exchange of probes required an average time of 4.4 seconds using the Robotiq adapter. All differences between probe adapters were statistically significant for time to swap probes (FIG. 13D).

A set of 5 ultrasound images of the phantom model were captured at a location where artery and vein features were evident with each of the probe adapters. The 5 images were taken at a repeated location with the probe orthogonal to the surface of the leg and then tilted±20° in the cranial-caudal and medial-lateral directions (FIG. 11). Referring now to FIG. 11, representative ultrasound images from repeatability testing with tissue phantom are shown. Ultrasound images of the phantom leg were captured to evaluate scanning repeatability of each probe adapter design at different angles (identified in figure), in accordance with various embodiments of the present disclosure.

Image variability was captured by tracking X and Y centroid coordinates of labeled features in images. Error in feature centroid coordinates were measures as standard deviation as a percent of total pixels in both the X and Y direction.

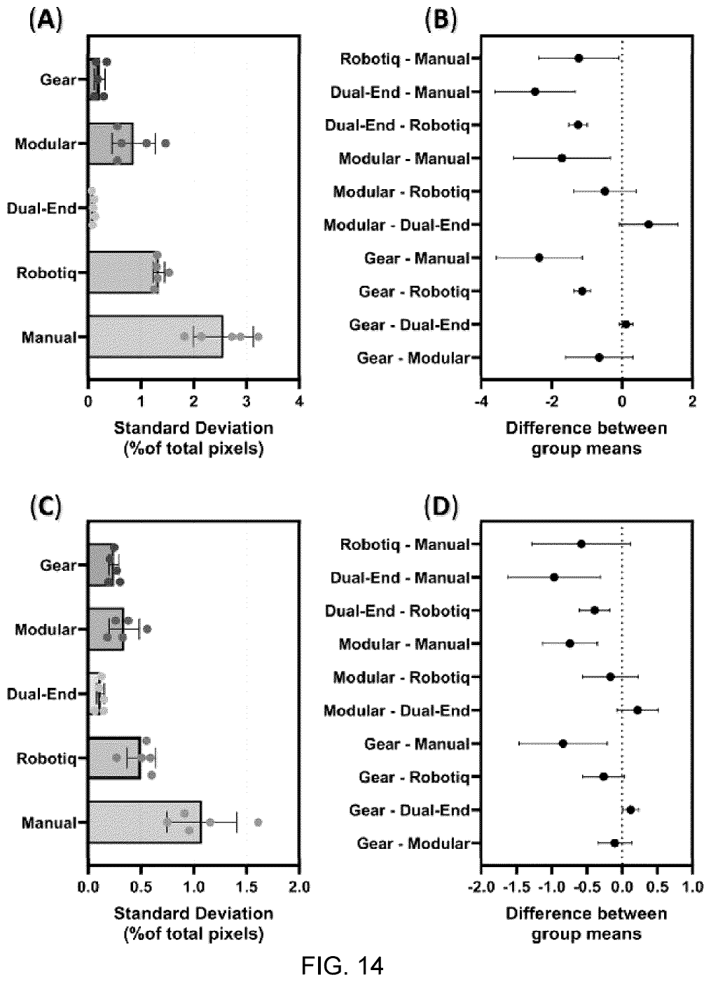

Referring now to FIGS. 14A, 14B, 14C, and 14D, ultrasound image repeatability results for each probe adapter are illustrated, in accordance with various embodiments of the present disclosure. Results are shown for the x-coordinate in FIGS. 14A, 14B and for the y-coordinate of the measured feature centroid (N=5) in FIGS. 14C, 14D. FIG. 14A, FIG. 14C illustrate the average standard deviation for each probe adapter and manual image acquisition. Error bars denote standard deviation for the 5 probe orientations used. FIG. 14C and FIG. 14D show 95% confidence intervals for the differences between each swap time group mean. Statistically significant comparisons are denoted by confidence intervals not crossing.

Overall, manual image acquisition demonstrated the highest standard deviation at 2.6% and 1.1% for the X and Y coordinates, respectively, shown in FIGS. 14A, 14C. Next highest standard deviation values were with the Robotiq gripper at approximately 1.3% in the X direction and 0.51% in the Y direction. Of the probe adapter designs, the Modular Design performed the worst with standard deviation values reaching above 0.87% for the X direction and below 0.34% for the Y direction. Conversely, the Dual-End Design resulted in the lowest standard deviation at 0.10% and 0.12% for the X and Y coordinates, respectively. Differences between a number of the probe adapter and manual image capture pairings were statistically significant for both the X and Y directions, shown in FIGS. 14B, 14D.

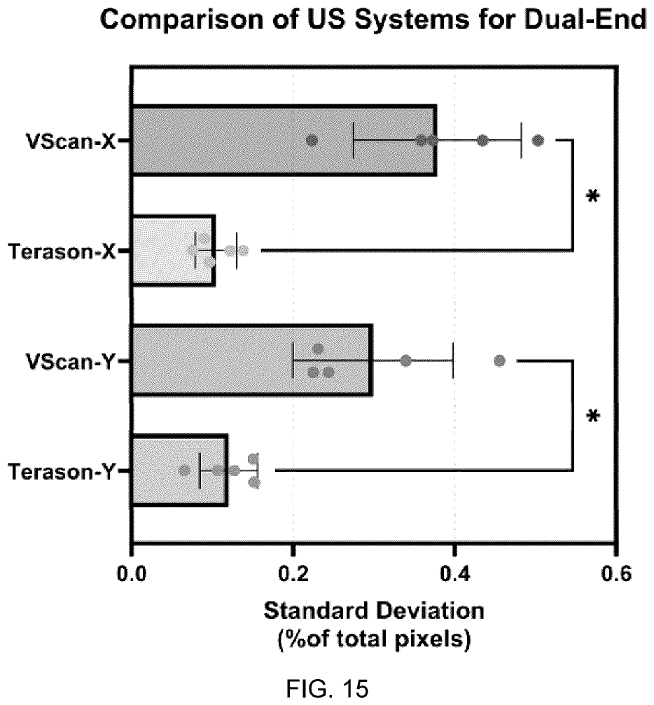

These results with the Dual-End Design were captured with the Terason ultrasound system. Additional images for this probe adapter design were taken using the curvilinear side of the Vscan Air™ ultrasound probe. This introduced additional variability likely due to the Vscan Air™ software's image capturing features. Instead of capturing single images a short 1-second video was captured by the Vscan Air™ software. The last frame of the video was exported and used to evaluate image repeatability. Images captured by the Dual-End Design using the Vscan Air™ probe resulted in statistically significant differences in both directions, with standard deviation of 0.38% and 0.30% for the X and Y coordinates, far exceeding the 0.10% X and 0.12% Y values using the Terason system (FIG. 15).

Referring to FIG. 15, comparison of ultrasound systems on the Dual-End Probe adapter are shown, in accordance with various embodiments of the present disclosure. Comparison of average standard deviation for both the centroid X and Y-coordinates for the VScan and Terason system are shown. Error bars denote standard deviation for the 5 probe orientations used. Differences between the ultrasound systems were statistically significance for both centroid coordinates (** p<0.01, ANOVA post-hoc Šídák test).

For each test, the end-adapters were scored so that the best performing design received a score of 100%, and the worst performing received a score of 0%. The remaining two designs received a calculated score that was normalized based on their performance relative to the results of the best and worst performing designs. This was done across the five performance measures for each end-adapter design. The Gear Design was best performing and thus received an overall score of 100%. However, the Dual-End Design scored similarly with a result of 96% The Modular Design performed worse at a score of 50%, comparable to the Robotiq gripper at a score of 48%.

Overall, all end-adapter designs were able to successfully reach the eFAST scan points and demonstrated a high rate of successful probe exchange. Differences between the designs were evident across other testing criteria, such as application force, where it was demonstrated that encasing the probe provided more reliable force application than by using the Robotiq gripper alone. The Gear Design and the Dual-End Design both provided a high degree of image repeatability with low variability of 0.24% and 0.11%, respectively, and outperformed other probe adapters, highlighting these as the highest performing across all evaluated criteria. The difference in performance between the Gear Design and the Dual-End Design was minimal, with both designs providing quick probe exchange times. For the Gear Design, size may limit its ability to reach a wider range of scan points in variable testing configurations. The Dual-End Design was intended for use with wireless ultrasound probe systems, having limited functionality for wired probes, with cables that would impede the 180-degree rotations required to swap probe types. Modifications address these challenges for both end adapter designs.

The Modular Design performed similarly to the Robotiq gripper, due to the additional time required to exchange probes and lower performance on image repeatability, relative to the other designs. The Modular Design adapter's worse performance across the evaluated testing criteria was likely due to the greater degree of physical exchange required by the modular components compared to the other more compact designs. However, the Modular Design adapter provided additional functionality through the ability to swap beyond just two probe styles to support additional attachments for therapeutic interventions, such as needle decompression for relieving tension pneumothorax injury.

The testing demonstrated that the robotic platform may be used to programmatically navigate to reach all the proper eFAST scan points. The end adapter designs allowed for the robotic arm to hold an ultrasound probe in reliable and repeatable manner. Automated ultrasound image capture and interpretation lowers the skill threshold required for medical assessment and provide accessible higher quality care for combat casualty care.

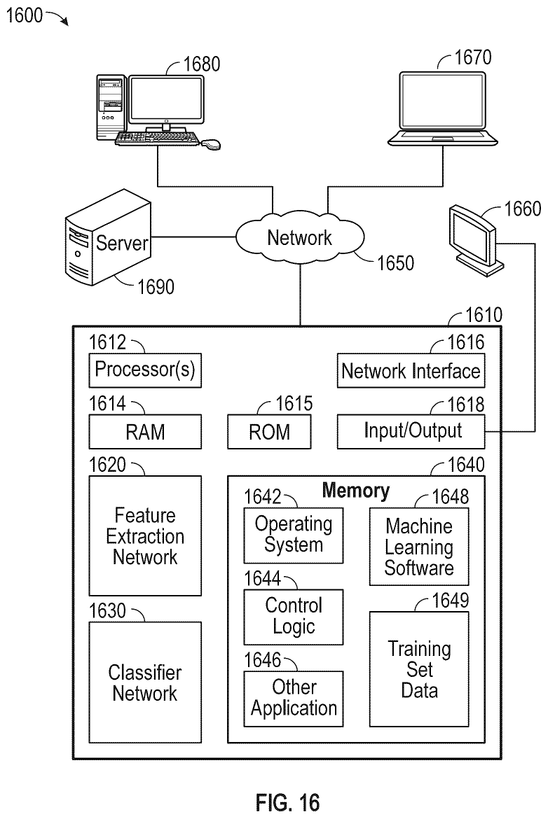

FIG. 16 illustrates a system block diagram 1600 including an example of a computing device 1610, such as the external video display device discussed above, that may be used in implementing one or more features of the disclosure, in accordance with embodiments of the present disclosure. Computing device 1610 may, in some embodiments, implement one or more aspects of the disclosure by reading and/or executing instructions and performing one or more actions based on the instructions. In some embodiments, computing device 1610 may represent, be incorporated in, and/or include various devices such as a desktop computer, a computer server, a mobile device (e.g., a laptop computer, a tablet computer, a smart phone, any other types of mobile computing devices, and the like), and/or any other type of data processing device. Further, as discussed previously, the video display device may comprise any machine configured to perform processing and/or calculations, may be but is not limited to a work station, a server, a desktop computer, a tablet computer, computing devices, a server farm, remote or wired machine, a personal data assistant, a smart phone, or any combination thereof. Moreover, as previously described, a server may be any server type such as, for example: a file server; an application server; web server; proxy server; an appliance; a network appliance; a gateway; a gateway server; a virtualization server; a deployment server; a Secure Sockets Layer Virtual Private Network (SSL VPN) server; a firewall; a web server; a server executing an active directory; a cloud server; or a server executing an application acceleration program that provides firewall functionality, application functionality, or balancing functionality.

As the software utilizes the processor and memory of an external video display device to function, the algorithm can be executed online or offline. The algorithms used herein has the built-in ability to configure video processing of the graphing processing unit (GPU) usage along with the computer processing unit (CPU) usage to utilize machine resources to execute the tasks described. It is understood that the described platform can be implemented using any computing technique, e.g., as a stand-alone system, a distributed system, within a network environment, etc. All processing of the algorithm model is preferably processed on the external screen device. In these embodiments, the software application does not rely on cloud services for image detection and deployment of algorithm.

Computing device 1610 may, in some embodiments, operate in a standalone environment. In others, computing device 1610 may operate in a networked environment. As shown in FIG. 16, computing devices 1610, 1670, 1680, and 1690 may be interconnected via a network 1650, such as the Internet. Other networks may also or alternatively be used, including private intranets, corporate networks, LANs, wireless networks, personal networks (PAN), and the like. Network 1650 is for illustration purposes and may be replaced with fewer or additional computer networks. A local area network (LAN) may have one or more of any known LAN topologies and may use one or more of a variety of different protocols, such as Ethernet. Devices 1610, 1670, 1680, and 1690 and other devices (not shown) may be connected to one or more of the networks via twisted pair wires, coaxial cable, fiber optics, radio waves or other communication media.

As seen in FIG. 16, computing device 1610 may include a processor 1612, RAM 1614, ROM 1615, network interface 1616, input/output interfaces 1618 (e.g., keyboard, mouse, display, printer, etc.), and memory 1640. Processor 1612 may include one or more computer processing units (CPUs), graphical processing units (GPUs), and/or other processing units such as a processor adapted to perform computations associated with machine learning. I/O 1618 may include a variety of interface units and drives for reading, writing, displaying, and/or printing data or files. I/O 1618 may be coupled with a display such as display 1660.

Memory 1640 may store software for configuring computing device 1610 into a special purpose computing device in order to perform one or more of the various functions discussed herein. Memory 1640 may store operating system software 1642 for controlling overall operation of computing device 1610, control logic 1644 for instructing computing device 1610 to perform aspects discussed herein, machine learning software 1648, training set data 1649, and other applications 1646. Control logic 1644 may be incorporated in and may be a part of machine learning software 1648. In other embodiments, computing device 1610 may include two or more of any and/or all of these components (e.g., two or more processors, two or more memories, etc.) and/or other components and/or subsystems not illustrated here. Moreover the device has a feature extraction network 1620 and a classifier network 1630.

As previously described, the memory may be any storage devices that are non-transitory and can implement data stores, and may compromise but are not limited to an optical storage device, a solid-state storage, hard disk drive, or any other magnetic medium, a ROM (Read Only Memory), a RAM (Random Access Memory), a cache memory and/or any other memory chip or cartridge, and/or any other medium from which a computer may read data, instructions, and/or code.

Devices 1670, 1680, and 1690 may have similar or different architecture as described with respect to computing device 1610. Those of skill in the art will appreciate that the functionality of computing device 1610 (or device 1670, 1680, and 1690) as described herein may be spread across multiple data processing devices, for example, to distribute processing load across multiple computers, to segregate transactions based on geographic location, clinician access level, quality of service (QoS), etc. For example, computing devices 1610, 1670, 1680, 1690, and others may operate in concert to provide parallel computing features in support of the operation of control logic 1644 and/or machine learning software 1648.

One or more aspects discussed herein may be embodied in computer-usable or readable data and/or computer-executable instructions, such as in one or more program modules, executed by one or more computers or other devices as described herein. Generally, program modules include routines, programs, objects, components, data structures, etc. that perform particular tasks or implement particular abstract data types when executed by a processor in a computer or other device. The modules may be written in a source code programming language that is subsequently compiled for execution, or may be written in a scripting language such as (but not limited to) HTML or XML. The computer executable instructions may be stored on a computer readable medium such as a hard disk, optical disk, removable storage media, solid state memory, RAM, etc. As will be appreciated by one of skill in the art, the functionality of the program modules may be combined or distributed as desired in various embodiments. In addition, the functionality may be embodied in whole or in part in firmware or hardware equivalents such as integrated circuits, field programmable gate arrays (FPGA), and the like. Particular data structures may be used to more effectively implement one or more aspects discussed herein, and such data structures are contemplated within the scope of computer executable instructions and computer-usable data described herein. Various aspects discussed herein may be embodied as a method, a computing device, a data processing system, or a computer program product.

### Example Animal Procedures and Manual Ultrasound Image Capture

In accordance with an example test, US scans were captured at eFAST scan sites using a swine model from three approved animal research protocols. FIG. 17 provides an overview of the animal study procedures and image capture timelines in this example. Ultrasound images were captured prior to splenectomy in live swine, as well as at two time points in euthanized swine (before and after eFAST injury induction). Each scan landmark in the diagram lists how US images were captured. The three approaches were manual US image capture, image capture using a real-time (RT) eFAST handheld application, and image capture using the robotic imaging platform. For all studies, images were captured immediately after instrumentation procedures and before laparotomy to remove the spleen, Scan #1 in FIG. 17). Each animal study was focused on different shock-related injuries, and splenectomies were performed to minimize the variability due to splenic contraction and autotransfusion. Since the spleen was removed in all protocols, no US scans were captured in the left upper quadrant, or LUQ, scan site. After the subjects were euthanized, two imaging rounds took place: before (Scan #2 in FIG. 17) and after inducing abdominal hemorrhage (AH), pneumothorax (PTX), and hemothorax (HTX) injuries at the respective scan sites (Scan #3 in FIG. 17).

For manual image capture, images in the thoracic region were captured using a linear array probe, and at the abdominal scan sites a curvilinear array probe was used (C5, Sonosite, Fujifilm, Bothwell, WA, USA), using a Sonosite PX (Fujifilm, Bothwell, WA, USA) US System. Images were captured for two different AI training applications: guidance and diagnostic AI models. For the diagnostic training dataset, thoracic US scans were captured as 10 s B-mode (brightness mode) clips or as 5 M-mode (motion mode) images, captured at multiple intercostal spaces. For guidance, 10 s B-mode clips were captured as a single swipe along all intercostal spaces of the thorax bilaterally. The abdominal scans were obtained at two locations: the right upper quadrant (RUQ), focusing on the kidney-liver interface, and the pelvic region (BLD), focusing on the areas around the bladder. For guidance image capture, 10 s region scans were captured in two motions: along the sagittal plane and along the medial plane. For diagnostic image capture, additional 10 s scans were captured while rocking the probe with the region of interest in view. All of these images were captured at three experimental timepoints, indicated as Scan #1, Scan #2, Scan #3 in Image Acquisition in FIG. 17.

### Data Processing

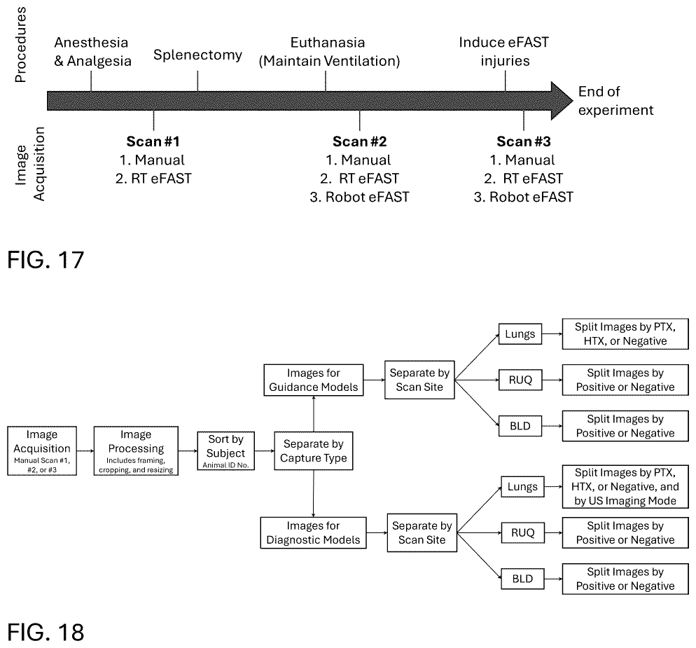

Ultrasound data from 36 pigs were exported from the US machine and sorted by experimental phase, subject ID, and scan point for both major scan types: guidance (scans along anatomical planes) and diagnostic (scans focused on organs, fluid accumulation sites), as diagrammed in FIG. 18. FIG. 18 provides an overview of ultrasound image dataset structure and processing for images captured in swine for eFAST AI model training, in accordance with various embodiments of the disclosure.

All ultrasound videos were split into frames, and individual images were cropped and resized to 512×512 pixels using the Image Processing Toolbox extension from MATLAB version R2023b (Math Works, Natick, MA, USA). Images were cropped to remove words and other artifacts on the US scans that the AI model may have focused on during training. The US scans were reshaped to a 512×512 pixel size to create a symmetrical image geometry at a high resolution to detect small injury features. Successful US AI models have also been developed for similar applications using this image input size. For guidance frames, datastore file types were created containing random samples of the data, with major anatomical features labeled with bounding boxes around them: ribs for thoracic scans, the kidney for RUQ, and the bladder for BLD. Once the labels were generated, images in which the feature was not obviously visible were removed from the dataset. The bounding box labels were exported from MATLAB as four coordinates: x, y of the top left corner, and x-length, y-length of the bounding box.

For diagnostic scans, images captured during the pre-splenectomy and pre-injury phases were preliminarily classified as negative for injury and the post-injury captures as positive for injury. Then, a file tree of all items was generated, which allowed the review of every entry. As part of data curation prior to training the AI models, all US scans were reviewed for the presence of injury and assessed for overall image quality score, injury severity (none, slight, positive), and the presence of motion artifacts (only applied to thoracic scans). Image quality evaluated whether the US scans could be used to diagnose an injury. In accordance with an example embodiment, a score of 1 corresponded to a poor image quality, with most frames captured at an incorrect location; a score of 5 corresponded to a high image quality captured at a proper eFAST scan point, where diagnostic status could be properly assessed. This was performed by two scorers who agreed on image quality scores for the initial frames to help standardize scoring and conferred to finalize data curation if disagreement occurred for any image. When selecting data for training the AI models, those with a signal quality score below 3 and thoracic scans with large motion artifacts were not included in the training datasets. Scans labeled as “slight” injury were maintained in the dataset as positive for injury.

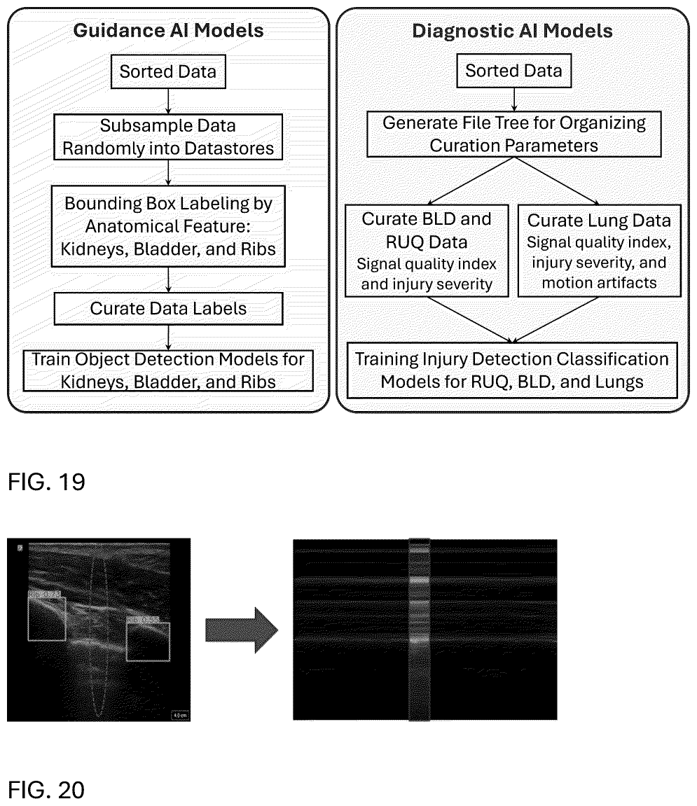

An overview of the AI model types used is shown in FIG. 19, in which a summary of data flow for cFAST AI model training is provided. For guidance models (diagram on the left in FIG. 19), data were subsampled, labeled, and then curated. More particularly, data was sorted; then subsample data was randomly stored into datastores; next, bounding box labeling by anatomical feature (such as kidneys, bladder, ribs) was performed (reference the red bounding boxes shown for the ribs image capture in FIG. 5A, for example); the data labels were assigned and curated; finally, object detection models were trained for the anatomical feature, such as kidneys, bladder, ribs. For diagnostic models (right diagram in FIG. 19), the sorted data were curated and then used for classification model training. More particularly, with regard to the diagnostic AI models, first data is sorted; a file tree is generated for organizing curation parameters; curate BLD and RUQ data (in this example) in which signal quality and injury severity is indexed and also curate lung data in which signal quality is indexed and injury severity and motion artifacts are logged; finally, injury detection classification models for RUQ, BLD, and Lungs are trained.

### Guidance AI Models

Once the data were labeled, the guidance AI models were trained using the YOLOv8 object detection architecture, with separate models tailored specifically for the detection of the kidneys (9449 labelled US images), bladder (7039 labelled US images), or ribs (44,736 labelled US images). The training process utilized the YOLOv8-S pre-trained model weights, default training parameters, and 100 epochs to provide ample opportunity for the models to learn and refine their predictions. To ensure robust model validation, a distinct dataset from subjects not used in training was reserved for the holdout testing of model performance. YOLOv8 was selected as the model architecture due to a variety of advantages when compared with other state-of-the-art object detection models. Primarily, this effort focused on the real-time application of object detection models with an eFAST-focused purpose. This meant that speed of prediction time was of high importance, even at the expense of slightly reduced accuracy. This narrowed the scope of possible models to be used to ‘single-stage’ architectures, where the single-stage model undertakes a single pass through of the image through the layers to determine the object location and class. Models like Faster R-CNN, for example, which can be more accurate, have a slower prediction time due to the image being processed into proposed regions of interest before being classified for objects. Moreover, when looking at single-stage models, YOLOv8 was amongst the fastest in frames per second, even beating out the single-shot detector (SSD) model and having only a slightly worse detection accuracy. Ease of use was also a driving factor for the use of YOLOv8 in this environment. The Python library ultralytics provides an API to allow for the seamless integration of YOLO models into existing software, for example.

For each guidance model trained, predictions were compared against the ground truth labels for the respective image, and Intersection-Over-Union (IOU) scores were calculated for each image. IOU is a metric for evaluating object detection models, calculated by dividing the area in which the predicted mask and ground truth mask overlap (intersection) by the total area covered by both masks (union). An IOU threshold of 0.5 is widely accepted in object detection applications as a standard for evaluating model performance, with scores at or above this threshold being acceptable. For kidney and bladder predictions, one object was expected for each frame, whereas for the thoracic image, two objects were expected. Regardless, for all predictions, the IOU score was calculated as an average across the entire image.

### Diagnostic AI Models

For diagnostic AI models, different approaches were used for the thoracic and abdominal regions. Each approach utilized the same YOLOv8 model architecture, except configured for classification for this use case. Diagnosis of injury in the abdominal region is regularly made from B-mode scans; as such, AI models were only trained using this type of imaging. In the thoracic region, due to the nature of lung sliding and how injuries present in ultrasound, M-mode images are a common means of distinguishing between injured and non-injured states. Diagnostic models trained for the thoracic region used two approaches: predictions from US-system-generated M-mode scans, or custom-generated M-mode images from a static hold in B-mode imaging mode. The latter approach is described below, followed by overall AI training procedures for the other scan points.

Diagnostic AI Models: Creating Custom Motion Mode Images from US Scans

For the development of diagnostic models focused on the thoracic region, first M-mode images were generated from the original B-mode US scans. This approach used a sequence of consecutive frames to create custom M-mode images. Each frame was processed through the guidance model for rib detection and, based on the predicted rib locations, the central point between the ribs was calculated. At this central point, a vertical slice was extracted from each frame as shown in FIG. 20. These slices were then concatenated to generate an image that closely resembled a genuine M-mode image. To ensure that a generated M-mode image was indicative of its diagnosis, the rib detection guidance model was used to filter out images without only two ribs visible. If a frame did not have exactly two ribs detected, that set of subsequent frames was not used for the M-mode creation process.

FIG. 20 provides an overview of how M-mode images were generated from B-mode frames using rib guidance AI models. Shown first in the left US image is a traditional B-mode ultrasound frame from which the guidance AI determined the location of the ribs (blue bounding boxes). A 3-pixel-wide region at the midpoint between the bound boxes (red dotted region) is selected across each frame to create a custom M-mode image, shown in the right US image.

An optimization process was conducted to determine the ideal set of parameters used to generate the images. These parameters included the number of frames per image, the width of the slice taken from each frame, the window stride between images, and the number of slices taken from each frame. The first two optimization parameters were concerned with the makeup of the generated M-mode images. For frames per image, 30, 90, and 150 frames per image were tested. In accordance with a specific example, images were captured from a video running at 30 frames per second, so these represent 1, 3, and 5 s capture windows. Three slice widths were also tested, these being 1-, 3-, and 5-pixel widths.

The remaining optimization parameters were focused on the generation of the training image dataset. The window stride parameter refers to the number of frames the model moves forward between images. For example, if using 30 images per generated M-mode and a stride of 15, one generated image will use frames 1-30, and the next will use images 15-45. The stride options used during the optimization were either 6 or 15 images, in a specific example. The final optimization parameter was the number of slices taken from each image, with either 1 or 3 slices being taken from each image. These parameters would affect both the number and makeup of images present in the training dataset.

These options produced 36 unique combinations of training parameters to be validated in the grid search using a YOLOv8 classification model trained for 100 epochs. After optimization, the resulting best parameters were as follows: 150-frame window size, 5-pixel slice width, 15-frame stride, and 1 slice taken per frame.

### Diagnostic AI Models: AI Models for Injury Identification

The diagnostic models were trained for injury detection at each eFAST scan site. For the abdomen, the AI models to identify AH injury were trained independently for the RUQ and BLD scan sites. For the thorax, two separate models were trained to predict if there was HTX, PTX, or no injury present, using either US-system-generated M-mode images or the custom generated ones as the input data. The dataset was split into 3 groups of 13 swine each to be able to perform the leave-one-subject-out (LOSO) cross-validation methodology. Each unique LOSO group was randomly generated from three research protocols and designated as a training, validation, or test set. Several AI model architectures were compared to develop AI models for each eFAST scan site. With the larger image dataset used in this study, these models needed to be retrained, and, for simplicity, utilized the same YOLOv8 architecture for image classification that was used for the AI guidance model development. The default training parameters were applied over a span of 100 epochs to allow for sufficient learning and refinement. Predictions were then tested on a holdout set of images from subject data not in the training data to test model performance. The best performing model from each scan site was then selected to be used in real-time testing.

### Real-Time Validation of AI Models

Real-time (RT) image capture was performed in three swine subjects completely separate from the dataset used to develop and test the underlying AI models. Each animal underwent imaging at the experimental timepoints shown in FIG. 17. Three real-time approaches were used: (i) RT eFAST application, which allowed for selection of a single scan site and capture of images while AI predictions for guidance and diagnostics occurred in RT; (ii) full handheld, manual cFAST examination, driven by AI guidance and diagnostic models; (iii) automated eFAST image capture using a robotic imaging platform equipped with computer vision, guidance, and diagnostic AI models. Each of these approaches is described in more details below.

Real-Time cFAST Application

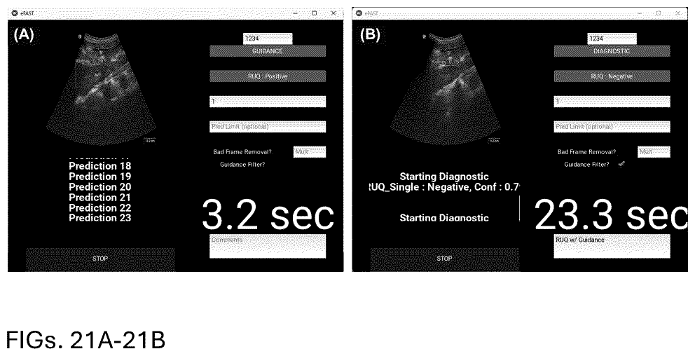

To enable the RT testing of models, a dedicated graphical user interface (GUI) was developed in Python using the Kivy library and designed to run on a laptop connected to the US machine via a Magewell USB Capture HDMI Gen 2 capture card (Magewell Electronics Co., Reading, PA, USA). The RT eFAST application allows users to input various experimental parameters, including subject identifier, scan mode (guidance or diagnostic), scan site (BLD, RUQ, M-mode, or RibsAI to generate M-Mode images), injury status, and number or duration of predictions as shown in FIGS. 21A, 21B, which provide an overview of the RT eFAST application. Additionally, the interface provides a comment section, with all inputs saved as a text file in addition to the prediction results from each individual scan. The best performing model in certain embodiments for each scan site and method that received the best blind test accuracy score was selected to be used in the real-time experiments. The trained model weights were packaged along with the GUI code to allow for the quick deployment of models and switching between models in real time. Users also have the option to select filtering methods that can be applied during the scan, as shown in FIG. 21B; these are further described in the next section.

Referring to example GUI shots shown in FIGS. 21A and 21B, in FIG. 21A, an GUI guidance AI model use is shown, while FIG. 21B illustrates a diagnostic AI model use, with guidance filtering active, is shown. These are representative screen shots shown for a RUQ scan site. The time refers to how long the application took to make predictions.

The RT eFAST application can be used for testing AI models in real time, as well as for data collection while performing the eFAST exam. The GUI allows the user to select relevant parameters for the operation and to start image capture. This in turn initializes the video stream and activates a thirty-second timer, which is displayed on the application. US imaging and RT predictions run for thirty seconds or until the specified number of predictions is reached, whichever comes first. While the scanning mode is active, the predictions and corresponding images are shown in real time, along with the prediction confidence scores. To ensure smooth operation, process threading may be employed to make predictions concurrently, preventing any interruption to the RT eFAST application's functionality. The system processed one frame at a time, waiting for each prediction to finish before loading the next frame.

As part of the data collection feature, the program can save all frames captured between predictions. A results folder was generated for every scan, containing subfolders for the saved intermediate frames and one for the frames used for the predictions, a CSV file listing model predictions with confidence scores, and a TXT file with user-input comments. For guidance scans, predicted images were stored with overlaid object detection boxes.

### Ultrasound Image Filtering Features

Several filtering options are available to the user while scanning: bad frame removal, guidance filtering, and the option to turn both of these on at the same time. The bad frame removal filtering option performs an analysis of each image to quantify the quality of the image based on intensity-based and texture-based features before predictions are made. To attain this functionality, a sample of 2000 images was taken from each scan site in the dataset and then analyzed using noise and pattern analysis to find some correlation between the ultrasound images labeled “bad” and quantifiable characteristics, such as average pixel intensity, the standard deviation of pixel intensity, entropy, or the signal-to-noise ratio. Images were labeled “bad” by two US operators based on the quality of the image and the ability to make a diagnostic prediction from the image. The metrics that indicated the strongest correlation to image quality were the average and standard deviation of pixel intensity, corresponding to the brightness and contrast of the images, respectively. Using this analysis, the most ideal values for brightness, contrast, and the signal-to-noise ratio were selected as the parametric floor to classify an image as a bad frame. The user also has the option to adjust the aggressiveness of bad frame removal from the GUI by entering a multiplier value to be applied to the bad frame parameters. Bad frame removal was only used for the RUQ and BLD sites, as the M-mode capture process required multiple seconds of undisturbed data capture, making bad frame removal not possible during this capture process.

In addition to bad frame removal, a guidance filter as a second filtering option was developed. For this process, streamed frames were passed through the guidance model for the designated scan site before any predictions were made. The guidance AI models evaluated each image for the identification of relevant anatomical features, such as two ribs, a bladder, or a kidney. If these features were not detected, the GUI bypassed the frame and moved on to the next available frame without making a diagnostic AI prediction. For the rib models, guidance occurred at the start of the scan. Once two ribs were identified, the GUI prompted the user to hold still for M-mode capture until the scan was complete, whether it was real or generated. For the RUQ and BLD models, guidance was applied before each prediction, with the model only proceeding if the appropriate anatomical features were detected in the image. When both filters were active, images were passed through bad frame removal first, followed by guidance filtering.

Manual eFAST Exam with AI Model Guidance

A python script was developed to test the guidance and diagnostic AI models during a full eFAST exam, recording the time taken to complete each scan point. The script prompted the operator to follow a scan order of upper-left thorax, lower-left thorax, upper-right thorax, lower-right thorax, RUQ, and BLD. For each scan point, the user prompts, model predictions, and the times taken to complete each scan were displayed in the command terminal. At the lung scan sites, the guidance model for lungs ran until it detected two ribs, and then prompted the user to stay in that location while it made three predictions using generated M-mode images, before telling the user to move to the next scan point. For RUQ and BLD, the user had to swap to the curvilinear transducer and then the guidance model ran continuously, only making a diagnostic prediction when the kidney or bladder was detected, until it reached 30 predictions. This imaging application was run in two modes: one in which the operator viewed the ultrasound screen during the exam, and a second “blind” scan where the user was unable to see the display. The manual eFAST exam with RT AI predictions was performed at the timepoints specified in FIG. 17.

### Automated Robotic US FAST Exam

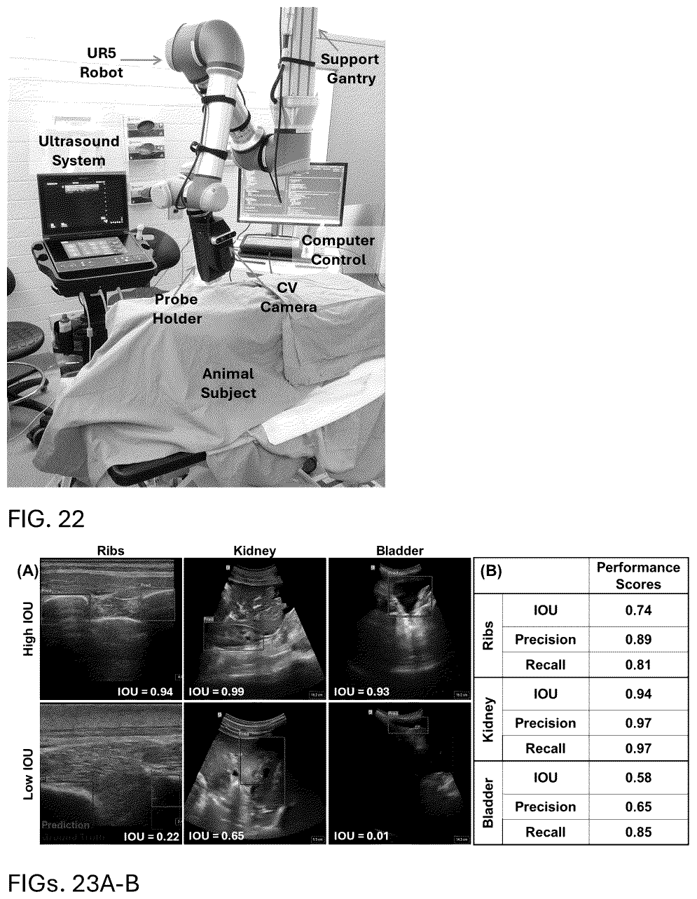

A UR5e robotic platform (Universal Robots, Odense, Denmark) is configured for semi-autonomous eFAST examination (FIG. 22). FIG. 22 provides an overview of an example robotic configuration for automated eFAST in swine. Relevant features of the setup are labeled to better explain the experimental setup.

The UR5e is programmed to navigate to eFAST scan sites using computer vision and stereo vision technology. Once at the scan site, the robotic arm is programmed to capture ultrasound images using a custom-made ultrasound probe holder to position the ultrasound probe and using integrated force feedback to apply the probe to the subject. Robotic navigation and image acquisition are further assisted by ultrasound-based guidance feedback that allows the robot to search a scan site at several positions until relevant anatomical features are in view of the image. Finally, the ultrasound images captured by the UR5e are evaluated for injury interpretation using the diagnostic AI models.

### Robotic Platform Configuration

The computer vision AI model detects the location of relevant scan sites on the subject's body using external image features. Ultrasound images are used to confirm the location of the relevant anatomical features for each scan site, and a fiducial target in the form of a circular color-coded sticker or other label is placed on the body of the subject at this location. The UR5e is programmed to travel around the body of the subject, capturing images using an Intel RealSense 435i camera (Intel, Santa Clara, CA, USA). Images are captured with and without the targets placed on the subject. eFAST scan sites are then labeled in MATLAB using the images that included targets. This process is repeated so that the image training dataset comprised images captured for two subjects. A computer vision model is then trained using YOLOv8s to accurately identify the color-coded stickers. Images of swine are also captured without stickers present to determine if the AI models could accurately identify scan sites without stickers present. Alternately, the computer vision models for detecting stickers at each scan site may be used. IOU scores were calculated for model predictions during the testing performed on the three swine subjects based on agreement between ground truth labeled sites and AI model prediction.

During testing, the UR5e is positioned over the subject at mid-torso using a hoist-lift structure (FIG. 22). The UR5e is programmed to capture four images of the top, left side, and right side of the pig using an Intel RealSense camera fixed to the end of the robotic arm, for example. For each image, the computer vision model detects the location of each scan site, providing the UR5c with real-world scan site coordinates for computer-vision enabled navigation. The model returned the pixel value of the center of the color-coded targets that were detected in each image. Next, with the inherent depth reading capabilities of the Intel RealSense camera associated with stereo vision technology, the real-world 3-dimensional location of the target relative to the lens of the camera is determined. The 3-dimensional location of the target is then transformed to the robot's coordinate system, allowing the robot to navigate to the scan site and apply the probe for image acquisition.

The quality of image acquisition is improved by using ultrasound image-based guidance feedback to scan a site, capturing multiple US images until an US image was acquired that could be used for proper diagnostic interpretation. For the abdominal sites, eight additional scan locations positioned in a circle equidistant apart at a 2.54 cm radial offset from the location of the original scan site, for example, were available for image capture. For the thoracic sites, the robot is programmed to scan linearly in intervals of 1.2 cm in the caudal direction before scanning another set of sites, following a line slightly offset in the same direction. This resulted in a total of 7 potential scan site positions for evaluation.

In addition to finding all the scan sites, radial positions, and linear positions on the subject, it was needed to ensure that the probe was oriented orthogonally and applied sufficient contact force to the surface to receive a clear ultrasound image. To do so, depths are measured at the detected scan point, so that the slopes of the measured surface can be used to calculate the correct roll, pitch, and yaw coordinates that would allow the robot arm to position the probe normal to the surface at each scan site. By accounting for the local curvature of the anatomy of the subject, adequate contact is sought between the surface of the ultrasound probe and the surface of the subject at each scan position. For the abdominal scan sites, a rocking B-mode scan is performed, where upon reaching an adequate position, the robot rotates to four different angles at a 5-degree offset relative to the scan site and collects a set of ultrasound frames at each different angle to pass to the diagnostic model. The set of ultrasound frames is acquired over a period of a tenth of a second for both the guidance and diagnostic scans, yielding between 5 and 7 frames.

Robotic cFAST (RoboFAST) Exam with AI Model Guidance

A set of three RoboFAST exams, each with a different set of criteria, in this example, are run on each of three experimental swine subjects at the two post-euthanasia timepoints (FIG. 17). All trained diagnostic and guidance AI models may be integrated into the RoboFAST algorithm to assess the robotic platform's capabilities and compare its performance to the manual eFAST exam performance. Upon detecting all scan sites and converting the pixel coordinates to coordinates relative to the origin of the robot, the robot starts the respective experimental run.

The first run, referred to as “Radar”, is a general eFAST exam where the robot scans both the original scan site and additional radial and linear positions until the guidance AI model returns that the proper organ or anatomy was present, indicating that a suitable location to run the diagnostic model was found. If no such detections occurred, the robot moves on to the next site without conducting a diagnostic prediction. However, when the guidance AI returns that the relevant object is detected, the diagnostic AI provides an injury prediction result for five consecutive frames. For the second run, referred to as “No Radar”, the robot performs a single image capture at the location where the colored sticker is detected. For the third experimental run, referred to as “All Radar”, the robot performs image capture at each scan site and all of the corresponding additional positions, running the diagnostic AI multiple times depending on how many positions at a site contained suitable locations. The plurality of what the diagnostic model returns then determines the prediction of the RoboFAST algorithm.

### Results-Guidance AI Performance

Referring now to FIGS. 23A, 23B, guidance AI performance for each anatomical location is shown, in accordance with various embodiments of the disclosure. FIG. 23A illustrates representative images for high and low IOU scores for rib, kidney, and bladder predictions. FIG. 23B illustrates testing performance scores for each anatomical guidance model for IOU, precision, and recall metrics.

For each guidance model trained, model performance was evaluated against a test dataset comprising images from subjects not included in the training data. Examples of high and low IOU scores are shown for each guidance model in FIG. 23A. The resulting average IOU scores varied across each model, with kidneys having the highest score at 0.94, followed by the ribs and bladder at 0.74 and 0.58, respectively, as shown in FIG. 23B. The precision and recall metrics were also strong for each guidance model, apart from precision for the bladder model, which was only 0.65 as shown in FIG. 23B. A higher false-positive rate due to the pixels being identified as bladder in the model's prediction but not in the ground truth image resulted in this lower score for the bladder model. Overall, each model was trained at variable performance levels and was able to correctly identify anatomical features to aid with proper eFAST US image acquisition.

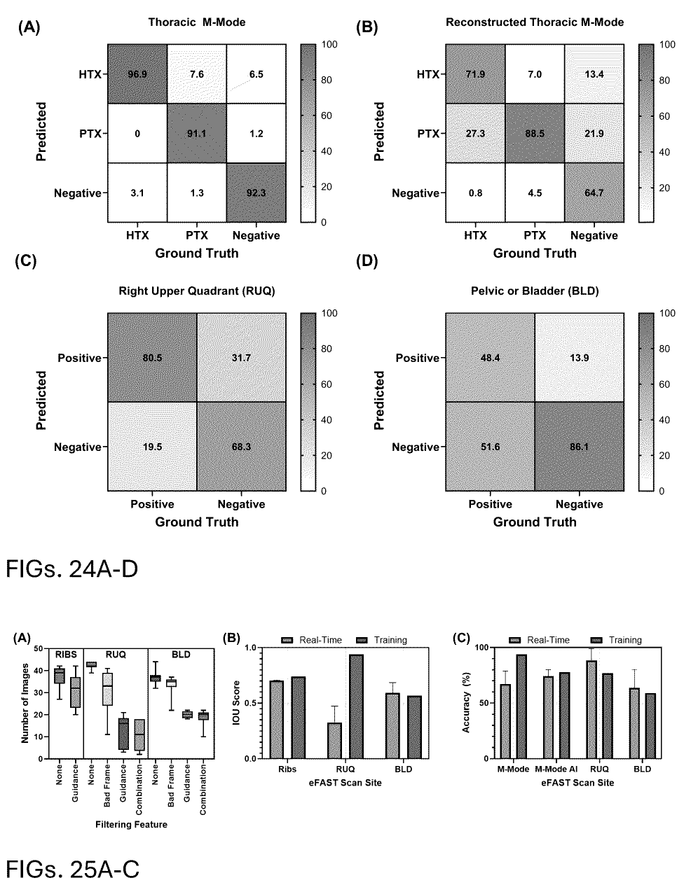

Referring now to FIGS. 24A-D, diagnostic AI confusion matrices for each diagnostic model are shown. FIG. 24A illustrates a three-class thoracic model using M-mode images. FIG. 24B illustrates a three-class thoracic model using M-mode reconstructed from B-mode frames. FIG. 24C illustrates a RUQ B-mode binary classification model. FIG. 24D illustrates a BLD B-mode binary classification model.

### Results-Diagnostic AI Performance

For thoracic diagnostic models, models were trained for both M-mode and generated M-mode diagnostic models (as described in Section 2.4.1). The M-mode diagnostic model predictions had a higher accuracy compared to the generated M-mode diagnostic models, at 0.94 vs. 0.78 accuracy, respectively. From the confusion matrix analysis, the generative M-mode models had a higher accuracy for the ground truth PTX predictions but identified 27% of the ground truth HTX images and 22% of the negative images as PTX (FIGS. 24A, 24B). Conversely, M-mode models had a slight bias toward HTX predictions, with 7.6% and 6.5% of the PTX and negative ground truth images being incorrectly identified as HTX-positive. Further RUQ and BLD diagnostic prediction models, binary in nature: positive or negative for abdominal hemorrhage, were developed. The RUQ models reached 0.77 accuracy but had a lower specificity metric of 0.68 compared to a higher recall of 0.80, hinting at slight bias toward positive predictions across the testing dataset shown in FIG. 24C. As for the BLD models, overall performance remained lower at 0.59 accuracy, with a much larger bias toward negative predictions in the testing dataset, as indicated by the confusion matrix and 0.49 recall metric as shown in FIG. 24D.

### Real-Time Model Performance

Real-time testing was conducted three different ways. The first used the RT eFAST application and was primarily used to evaluate the AI guidance and diagnostic model performance at each scan site, along with the utility of different filtering approaches. The other two approaches were the manual, handheld eFAST exam with AI model feedback and RoboFAST. Both of these approaches allowed for a full eFAST exam to measure the timing of the procedures and how the AI models synergized with various image acquisition approaches.

Evaluation of the Real-Time cFAST Application

Starting with the RT eFAST application, reference is made to FIGS. 25A-25C. FIGS. 25A, 25B, 25C illustrate an evaluation of a real-time eFAST application, in accordance with various embodiments of the disclosure. At FIG. 25A the total number of images captured at each scan location for a set 30 s capture window for various pre-processing filter methods is illustrated. Averages are shown along with the size of the box highlighting the 25th and 75th quartiles, while error bars denote minimum and maximum values. FIG. 25B illustrates performance IOU results for AI-guided manual US image capture compared to test performance results during model training. FIG. 25C illustrates diagnostic accuracy of real-time image capture compared to test accuracies during model training for each scan location. Mean values are shown with error bars denoting standard deviation.

It can be seen that the different filtering methods impacted the number of images that were captured at each scan site during a 30 s data capture window as shown in FIG. 25A. For ribs, on average, six less images were captured when using the guidance filter (approximately 37 vs. 31 images). Bad frame filtering was not applicable at this scan point due to M-mode capture needing to be continuous and not interrupted by frame removal procedures. The effects were more noticeable with RUQ and BLD, where bad frame filtering reduced the number of images by 12 and 3 images, respectively, while guidance filtering reduced the number of images by 30 and 16 images, respectively. Compounding these approaches reduced the number of images sent to the diagnostic models by 32 and approximately 18 images, respectively.

Next, how the guidance models performed using the RT eFAST application was evaluated. This was undertaken without any filtering methods applied to obtain an overall IOU performance metric for each scan site as shown in FIG. 25B. In real time, performance decreased for ribs (0.70 real time vs. 0.74 training) and more substantially for the RUQ (0.33 real-time vs. 0.94 training), while BLD performance slightly increased (0.59 real time vs. 0.57 training). In terms of diagnostics, the effects of these filters on overall diagnostic accuracy were minimal, so the averaged diagnostic accuracy results comparing training performance are shown in FIG. 25C. Performance was comparable to training data, with the exception of the M-mode thoracic model, which had a reduced accuracy of 0.67 compared to 0.94 during model training.

### RoboFAST Evaluation

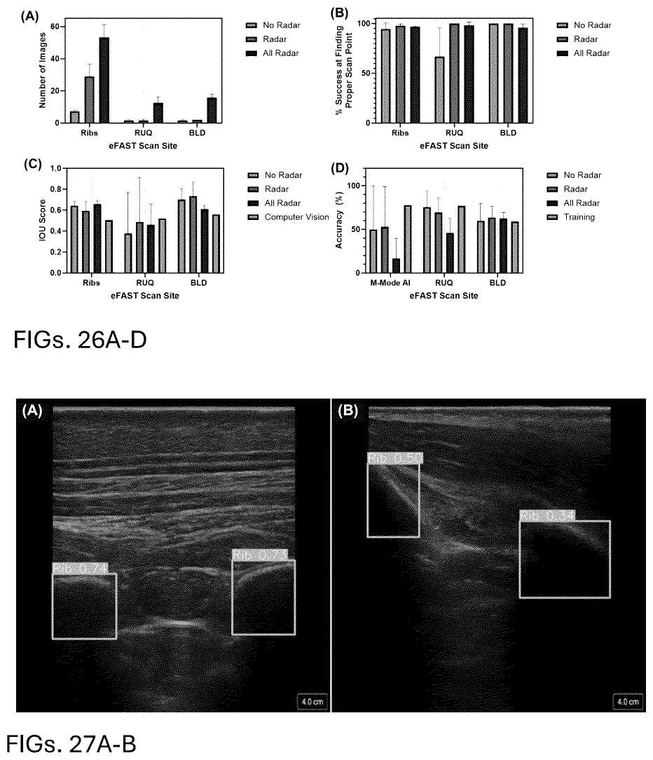

Referring now to FIGS. 26A-D, performance evaluation in swine is illustrated. In FIG. 26A, the number of images captured with each imaging modality with the robotic imaging platform is illustrated. In FIG. 26B, the overall success of RoboFAST in finding an US image to send to diagnostic AI models for each scan point and imaging modality is shown. FIG. 26C illustrates IOU performance results for guidance AI models using No Radar, Radar, and All Radar modalities; computer vision IOU scores for identifying scan sites are also shown for ribs, RUQ, and BLD positioning. FIG. 26D illustrates diagnostic accuracies for each scan modality compared to diagnostic model blind test accuracies during training. Averages are shown and error bars denote standard deviation across triplicate swine subjects throughout.

The robotic imaging platform relied on a computer vision model to identify each eFAST scan site automatically. The IOU scores for these predictions across scan sites were as follows: 0.51, 0.52, and 0.56 for ribs, RUQ, and BLD, respectively, as shown in FIG. 26C. For US image capture, three approaches were used to capture images, as described above, using the Robotic eFAST (RoboFAST) exam with AI model guidance: Radar, No Radar, and All Radar modalities. First the effects of the various methods on the total number of images captured was evaluated as shown in FIG. 26A. As anticipated, the All Radar approach captured the most images for each scan site, while No Radar and Radar had similar numbers of images for the RUQ and BLD scan sites. Next the overall success of each scan site across the three swine subjects was quantified, where success is defined as at least one image being captured that could be used for diagnosis as shown in FIG. 26B. All approaches had high performance, except for the RUQ/No Radar approach at 67% success. Factoring this in, Radar and All Radar had similar performance levels for this evaluation criterion. The guidance model IOU performance scores were similar for each RoboFAST imaging modality, with BLD having the highest IOU scores and RUQ performing the worst and having the highest subject variability as shown in FIG. 26C.

Lastly, diagnostic model performance was evaluated. The All Radar modality resulted in the lowest accuracy for the M-mode thoracic AI (16.5%) and RUQ (46%) models, as shown in FIG. 26D. Radar and No Radar performed similarly at each scan site. Compared to the test results obtained during model training, BLD and RUQ were comparable to the RoboFAST captured accuracies, while RoboFAST severely underperformed for the thoracic scan sites. This may be a result of the robotic imaging platform experiencing difficulty reaching the proper thoracic scan site where pleural space was present, as shown in the representative US images captured during RoboFAST, as shown in FIGS. 27A, 27B in which RoboFAST thoracic US images are illustrated. Representative US images captured by the robotic platform with pleural space in view in FIG. 32A and not in view in FIG. 27B.

### Timing Comparison Between Handheld FAST Application and RoboFAST

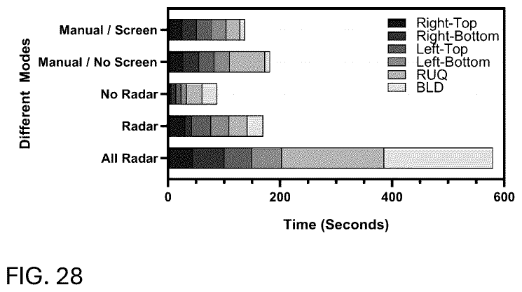

FIG. 28 illustrates a summary of eFAST image capture times. Results are shown for all scan sites evaluated for each configuration of the manual AI-guided and automated robotic image platform. Average results are shown for each scan site across triplicate animal experiments.

Ultimately, the overall time required to complete two RT cFAST imaging methodologies was compared as shown in FIG. 28. Instead of the RT cFAST application, the AI models were configured for use in sequence across six total scan locations to mirror how the images were captured with the robotic imaging platform: (i) right thoracic top and (ii) bottom, (iii) left thoracic top and (iv) bottom, (v) RUQ, and (vi) BLD, described above, matching the number of scan sites used during RoboFAST testing. The timing of image capture by the end user having or not having the US screen visible (only relying on AI predictions and instructions to move to the next scan site), which resulted in a slightly longer time on average with no screen visible compared to when the screen was present (138 s manual, screen vs. 183 s manual, no screen), was evaluated. The RUQ scan site was most impacted by not looking at the US screen, as most captured images were excluded by the guidance filter. For the robotic imaging platform, the No Radar modality was the quickest (87 s), with rapid thoracic image capture compared to the slower Radar image capture (170 s), and the overall slowest All Radar modality (580 s).

### Applications in View of the Above

As ultrasound technology becomes smaller and more portable, its potential utility in emergency medicine widens. Pre-hospital triage by US imaging may be possible if the challenges of imaging can be reduced so that less-skilled personnel can perform initial triage assessments. This is especially true for military medicine, where triage decisions in the battlefield must prioritize limited evacuation opportunities in scenarios where air evacuation is not readily available, as has been the case in recent conflicts. The AI-driven tools described herein demonstrate how US imaging can be simplified to lower the skill threshold for triage on future battlefields or in other civilian emergency situations.

Of interest is the automation of image acquisition techniques. Guidance object detection AI models were built using a YOLO model architecture, which was further tuned for use with swine datasets. Performance was mixed in the real-time implementation of these models, with BLD and RUQ underperforming compared to rib detection models. Nonetheless, this still highlights how guidance models could assist with real-time scanning. These models can be used as a filter during manual scanning to exclude all frames in which key anatomical features are not present. Additionally, they may be used to provide autonomous feedback to robotic image acquisition platforms to acquire images with evident anatomical features that are required for proper diagnostic interpretation. Refined models may ensure that not only anatomical features are present in the image, but also that the ideal anatomical features for diagnostic determination are identified. For instance, models confirmed the presence of two ribs in each image so that the pleural space between the ribs can be evaluated for diagnosis. However, if the probe is not oriented correctly, the pleural space cannot be seen, making injury identification impossible. Additionally, the model confirms the presence of a kidney in each image to evaluate RUQ scan sites. It is noted that since fluid often pools around the edges of the kidney, guidance models could confirm that the edges of the kidney are in view so that images used for diagnostic interpretation capture the area most likely to demonstrate evidence of injury.

Diagnostic AI models may be further refined prior to real-time application. US image sets expanded to more than 35 swine subjects allow for use a YOLOv8 image classification model. A likely reason for the difference in model performance, in which the guidance models were consistently more accurate compared to the new diagnostic models, is that the guidance models were required to identify anatomical landmarks, while the diagnostic models were tasked with the more difficult task of interpreting nuanced changes in variable injury sizes. Additional image curation, robust model architecture, and rigorous model fine-tuning will further improve AI training performance and the use of these models for real-time image interpretation. As for the methods of exploring model architectures, deep learning models used for segmentation can be applied to localize features of injury to help the models attribute the presence of fluid around the bladder, resulting in positive classifications. Long short-term memory (LSTM) networks used in video analysis can be explored to give the models more context on the appearance of variable injury sizes when making predictions on sequential images. Lastly, adding filters or pre-processing techniques with the purpose of amplifying relevant areas of the bladder can be tested for model training to help differentiate features between classifications.

AI models were evaluated in real time, with and without a robotic imaging platform, highlighting example, different end-user applications of this technology. The handheld manual AI-guided application had faster performance, but still requires a user to position the probe in the right location. Filtering approaches were used to exclude images that were not suitable for diagnostic evaluation, which resulted in the exclusion of a large number of images from the diagnostic pipeline. Image filtering is important for automated image acquisition in a handheld format, as less-experienced users may place the ultrasound probe at incorrect positions that may not have been included in diagnostic AI training datasets, resulting in a higher likelihood of incorrect diagnostic predictions. An alternative to making diagnostic models more robust to handle these irregular images is that filtering applications can prevent these images from impacting diagnostic predictions. Large datasets paired with modified diagnostic models will help the development of these filters and manual AI-guided cFAST image capture techniques.

For the robot image capture platform, different configurations had a wide impact on the speed of performing an eFAST examination, a result of the number of images being captured and the need to ensure that a proper eFAST viewpoint is captured at each scan site. A robot's limited range of motion may be challenged by the deeper angles required to image the RUQ or the lower thorax, where HTX injuries are often identified. This may be affected by the robot image capture platform configuration, such as the bulkiness of the platform and poor clearance with the table on which the subject was placed. Guidance models performed as expected; diagnostic model accuracies for the thoracic scan sites may be improved by gradual movement and better tracking of the proper direction to move across the thoracic cavity. Conversely, the RUQ and BLD had similar accuracy to the testing results of the diagnostic models, providing evidence of the utility of robotic mechanisms to automate image capture.

The utility of the handheld and robotic cFAST imaging platforms differ greatly in their potential applications. A large robotic system is not feasible in all pre-hospital settings but could be envisioned at a site for processing mass casualty scenarios, for automated triage assessment in a hospital, or military applications. Less human support is needed once the technology is further refined, so a more automated design can potentially streamline casualty in-processing. In direct contrast, the handheld tool, if paired with small, portable US devices, could be deployed in ambulatory civilian care or military care near the point of injury. While the technology will still require the user to manipulate the technology to proper positions, additional guidance measures in the software application can further lower the skill threshold during real-time deployment.

The example and alternative embodiments described above may be combined in a variety of ways with each other without departing from the invention.

Embodiments of the invention have been described to explain the nature of the invention. Those skilled in the art may make changes in the details, materials, steps and arrangement of the described embodiments within the principle and scope of the invention, as expressed in the appended claims.

While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method for obtaining one or more ultrasound images for use in an eFAST examination, the method comprising:
moving a robotic arm along a patient's body while sending images to a controller;
analyzing the images for anatomical landmarks to identify measurement areas;
sending instructions from the controller to the robotic arm to direct the robotic arm to the measurement areas; and
pressing an ultrasound transducer against the patient's skin to capture an ultrasound image upon reaching the measurement area.

2. The method according to claim 1, further comprising applying ultrasound gel on the measurement area and optionally having the application triggered by the controller.

3. The method according to claim 1, further comprising repeating the method for each measurement area.

4. The method according to claim 1, further comprising
changing the ultrasound probe from a linear probe to a curvilinear probe or vice versa, or
rotating a probe adapter from a linear probe to a curvilinear probe or vice versa.

5. The method according to claim 1, where a probe adapter attached to the robotic arm is selected from one or more of a Gear Design, a Dual-End Design, and a Modular Design.

6. A system comprising:
a robotic arm;
a probe adapter configured to attach to the robotic arm and to hold an ultrasound transducer, the probe adapter having:
a pressure sensor and/or a spring mechanism for controlling an applied pressure of the ultrasound transducer against a patient's skin; and
an optional gel dispenser configured to deliver ultrasound gel to the patient's skin; and
a controller in electrical communication with the robotic arm and configured to control the movement of the robotic arm and the capturing of ultrasound images to locate the ultrasound transducer in the measurement areas.

7. The system according to claim 6, further comprising a linear ultrasound transducer and/or a curvilinear ultrasound transducer.

8. The system according to claim 7, where the probe adapter is selected from one or more of a Gear Design, a Dual-End Design, and a Modular Design.

9. The system according to claim 8, where the controller includes a model trained using images of the bodies of different individuals and/or ultrasound images of different individuals to identify measurement areas for obtaining ultrasound images for an eFAST examination.

10. The system according to claim 7, where the controller includes a model trained using images of the bodies of different individuals and/or ultrasound images of different individuals to identify measurement areas for obtaining ultrasound images for an eFAST examination.

11. The system according to claim 7, where the ultrasound images include measurement areas from the pelvic area through the shoulder area including organs and bones.

12. The system according to claim 6, where the probe adapter is selected from one or more of a Gear Design, a Dual-End Design, and a Modular Design.

13. The system according to claim 12, where the controller includes a model trained using images of the bodies of different individuals and/or ultrasound images of different individuals to identify measurement areas for obtaining ultrasound images for an eFAST examination.

14. The system according to claim 12, where the ultrasound images include measurement areas from the pelvic area through the shoulder area including organs and bones.

15. The system according to any one of claim 6, where the controller includes a model trained using images of the bodies of different individuals and/or ultrasound images of different individuals to identify measurement areas for obtaining ultrasound images for an eFAST examination.

16. The system according to claim 6, where the ultrasound images include measurement areas from the pelvic area through the shoulder area including organs and bones.

17. The system according to claim 16, where the model uses images of the patient's skin to determine where the probe adapter is located.

18. The system according to claim 16, where the model uses ultrasound images of the patient to identify where the probe adapter is located based on the organs and/or bone structure present in the ultrasound image.

19. The system according to claim 15, where the model uses images of the patient's skin to determine where the probe adapter is located.

20. The system according to claim 19, where the model uses ultrasound images of the patient to identify where the probe adapter is located based on the organs and/or bone structure present in the ultrasound image.

21. The system according to any one of claim 15, where the model uses ultrasound images of the patient to identify where the probe adapter is located based on the organs and/or bone structure present in the ultrasound image.

22. The system according to claim 6, further comprising:
a camera attached to the robotic arm and directed towards where the probe adapter is located to visualize a location of the robotic arm relative to the patient; and
a display to show images captured by the one or more of the camera and the ultrasound transducer to provide a control interface for the system.
